(19) United States
(12) Patent Application Publication
(10) Pub. No.: US 2026/0258405 A1
(43) Pub. Date: Sep. 3, 2026

(54) CAPLESS AND TAILLESS THERAPEUTIC EXOGENOUS mRNA AND METHOD TO PRODUCE THE SAME

(71) Applicant: The University of South Alabama, Mobile, AL (US)

(72) Inventors: Viktor Solodushko, Mobile, AL (US); Brian Fouty, Spanish Fort, AL (US)

(73) Assignee: The University of South Alabama, Mobile, AL (US)

(21) Appl. No.: 18/867,690

(22) PCT Filed: May 19, 2023
(86) PCT No.: PCT/US2023/022991

§ 371 Date: Nov. 20, 2024

Related U.S. Application Data
(60) Provisional application No. 63/344,299, filed on May 20, 2022.

(51) Int. Cl. C12N 15/11

(52) U.S. Cl. CPC C12N 15/11; C12N 2310/531; C12N 2330/30

(57) ABSTRACT

A biologically functional polynucleotide in the form of therapeutic exogenous mRNA having hairpins in lieu of a cap and poly(A) tail and method of producing the same is provided. The design of a biologically functional polynucleotide allows for a one step method of production. The target mRNA vector or chemically synthesized mRNA molecule of the biologically functional polynucleotide includes internal translation initiation site and delivered gene sequence. Additionally, the target mRNA vector is designed to form stable hairpins at both the 5′ end and 3′ end, wherein said hairpins comprise at least four paired nucleotides in the stem and linked to the closest end with no more than two unpaired nucleotides.

### CROSS-REFERENCE TO RELATED APPLICATIONS

This application claims the benefit of International Application PCT/US23/22991, filed on May 19, 2023, which claims priority to U.S. Provisional Application Ser. No. 63/344,299, filed on May 20, 2022, wherein said applications are incorporated herein in their entirety by reference.

### FIELD OF THE DISCLOSURE

The subject matter of the present disclosure refers generally to synthesizing therapeutic exogenous mRNA having hairpins in lieu of a cap and poly(A) tail.

### BACKGROUND

The success of the mRNA vaccines in controlling the SARS-COV-2 pandemic has confirmed the efficacy of mRNA immunizations, and since the first approval of mRNA vaccines for the SARS-COV-2 pandemic by the FDA in 2021, scientist have been equipped with much more information regarding the design and synthesis of mRNA for vaccines as well as developed a better understanding as to the production costs for such vaccines. Currently, mRNA that is synthesized for use as a therapeutic or a vaccine is usually transcribed in vitro using a DNA template and is capped and polyadenylated. The 5′ cap and 3′ poly(A) tail are important not only for initiating protein translation, but also for protecting against 5′ and 3′ exonucleases that can quickly degrade the mRNA transcript. Unfortunately, the 5′ cap is the most expensive component of exogenously produced mRNA, accounting for about 45% of the raw material costs.

A major advantage of exogenous mRNA-based vaccines is that they can be produced less expensively and faster than conventional vaccines, such as subunit, live-attenuated, and inactivated viruses. Further, exogenous mRNA-based vaccines also avoid safety issues intrinsic to working with live viruses, resulting in a simpler downstream purification process and more rapid manufacturing when compared to conventional vaccine manufacturing methods. In addition to mRNA-based vaccines, other therapeutic exogenous mRNA-based technologies have seen a recent surge in interest in the biotech field. This is especially true in situations where short-term protein expression is a required trait in a therapeutic agent since therapeutic exogenous mRNA function and degradation occur via the same mechanisms that degrade that cause endogenous mRNA to function and degrade. In addition to vaccines, biotechnologies to which therapeutic exogenous mRNA might be especially relevant include protein replacement, gene therapy, and cancer immunotherapy. Exogenous mRNA may also be useful as a therapeutic agent for the treatment of clinical diseases.

As previously mentioned, current synthesis methods of exogenous mRNA-based biotechnologies also incorporate cap and tail structures. This is largely because nearly all endogenous eukaryotic mRNA contains a 5′ cap structure and a 3′ chain of adenosine nucleotides (poly(A) tail) added during RNA processing. Though both the capping and the addition of the long poly(A) tail to the eukaryotic mRNA are critical for proper function and stability of current exogenous mRNA-based biotechnologies, both the capping and addition of the tail require additional steps that slow production of exogenous mRNA-based biotechnologies and add to the cost to manufacture said vaccines. Redesigning mRNA transcripts so that they retain their function and stability without the requirement for a cap and a lengthy poly(A) tail would reduce cost, increase speed, and improve yield of in vitro mRNA production. The cap is required not only to initiate translation, but also to protect the 5′ end of the mRNA transcript from exonucleases while the poly(A) tail is necessary to protect the 3′ end from exonucleases. Any alternative to the cap and tail will need to accomplish these tasks.

One potential solution to replace the cap's role in initiating translation is to use internal translation initiation site which can range from a modified single nucleotide to a sequence that is hundreds of modified or unmodified nucleotides long and located in the middle of a polyribonucleotide. Known examples of internal translation initiation include, but are not limited to, IRES-stimulated translation initiation, 5′ UTR m6A-mediated translation initiation, YTHDF1-mediated translation initiation, Ribosome shunting, and Repeat-associated non-AUG (RAN) translation. However, using such methods to initiate translation in a transcript that lacks a 5′cap and 3′poly(A) tail still requires a way to protect the 5′ and 3′ ends from exonucleases.

Accordingly, there is a need in the art for an in vitro synthetized mRNA transcript that can be functional and stable without a 5′ cap and/or 3′ poly(A) tail. Further, there is a need in the art for therapeutic exogenous mRNA-based technologies that may be produced in vitro in a single step as to increase production efficiency and reduce production costs.

### SUMMARY

A therapeutic exogenous mRNA having hairpins in lieu of a cap and poly(A) tail and method of producing the same is provided. In one aspect, the biologically functional polynucleotide and method for production may be used to speed up the production of vaccines. In another aspect, the biologically functional polynucleotide and method of production may be used to decrease the cost of vaccines and treatments for transient disease/injury. In yet another aspect, the biologically functional polynucleotide and method of production may be used to increase the yield of RNA when same amount of row materials is used for its production. Generally, the biologically functional polynucleotide and method of production of the present disclosure are intended to be used as an alternative method for the design and production of therapeutic exogenous mRNA (both linear and circular). Additionally, the design of a biologically functional polynucleotide and its method of production allow for a one step method of production as opposed to some multistep method of production that the more traditional cap and tail therapeutic exogenous mRNA molecules require.

In a preferred embodiment, the biologically functional polynucleotide is mRNA that comprises a target mRNA designed to form hairpins at both the 5′ end and 3′ end, wherein said hairpins have no unpaired nucleotides in the stem and between the stem and the end. The hairpins of said biologically functional polynucleotide are preferably formed by the pairing of no less than 4 complementary base pairs, resulting in a segment of double stranded mRNA at the 5′ and 3′ ends. Each hairpin contains at least one loop, wherein said at least one loop is comprised of no less than 2 unpaired nucleotides. Further, the loop is a sequence that does not form stable double stranded RNA within the given hairpin, but can form connections with other sequences within the molecule without disrupting the given hairpin. The target mRNA preferably comprises an mRNA transcript or chemically synthesized mRNA molecule that includes an internal translation initiation site incorporated upstream of a transgene. In a preferred embodiment, the internal translation initiation site is internal ribosome entry site (IRES) and is incorporated into said mRNA transcript or said chemically synthesized mRNA molecule within the 5′ untranslated region (5′ UTR) of said mRNA transcript or chemically synthesized mRNA molecule. The hairpins are contiguous with the rest of mRNA at the 5′ end and 3′ end and include, but are not limited to, single hairpins, double hairpins, and triple hairpins, wherein said double hairpins and triple hairpins have zero or no more than two unpaired nucleotides separating each individual hairpin. The terminal hairpins can also serve as another important structured part in mRNA molecule (part of structured IRES, hairpin-based protein binding sites, etc.) playing additional roles in mRNA translation and stability. Therefore, embodiments of the biologically functional polynucleotide may comprise a target mRNA having a plurality of different types of hairpins without departing from the inventive subject matter described herein. Further, the hairpin at the 5′ region is operably linked to the internal translation initiation site by a linker nucleotide sequence that can vary in sequence and number whereas the hairpin at the 3′ region is operably linked to the open reading frame (ORF) of the delivered gene by another linker nucleotide sequence that may also vary in sequence and number. Additionally, the linker nucleotide sequences to which the hairpins are operably linked may differ in sequence and number.

The DNA sequence used to create the target mRNA sequence is first inserted into a DNA source (plasmid DNA, genomic DNA, synthetic DNA, PCR product, etc.) to create a DNA template, wherein said DNA template is configured to encode the RNA transcripts containing the target mRNA having the internal translation initiation incorporated upstream of a delivered gene sequence and contiguous with hairpins at both ends. In vitro transcription is then accomplished using T7 RNA polymerase; however other RNA polymerases, such as SP6, T3, and others, may be used without departing from the inventive subject matter described herein. This results in the target mRNA vector harboring the internal translation initiation upstream of a delivered gene sequence. Further, because the DNA template is configured to produce an mRNA transcript with an internal translation initiation incorporated therein and having hairpins contiguous with both the 5′ and 3′ ends, no cap or poly(A) tail must be added in subsequent steps, allowing the creation of stable and functional therapeutic exogenous mRNA in a single step. Alternatively, the terminal hairpin can be added to mRNA at the 5′ end only. In this case, a poly(A) tail may be needed at 3′ end for mRNA protection and proper functionality. Likewise, the terminal hairpin can be added to mRNA at the 3′ end only. In this case, a cap may be needed at 5′ end for mRNA protection and proper functionality. Natural and modified ribonucleotides (or their combinations) may be used for the synthesis of the target mRNA. Alternatively, the target mRNA vector may be chemically synthesized without a DNA template to produce therapeutic exogenous mRNA in a single step.

The foregoing summary has outlined some features of the biologically functional polynucleotide and method of production so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the biologically functional polynucleotide and method disclosed herein. Those skilled in the pertinent art should also realize that such equivalent designs or modifications do not depart from the scope of the biologically functional polynucleotide and method of the present disclosure.

### DESCRIPTION OF THE DRAWINGS

These and other features, aspects, and advantages of the present disclosure will become better understood with regard to the following description, appended claims, and accompanying drawings where:

FIG. 1 is a diagram illustrating a biologically functional polynucleotide embodying features consistent with the principles of the present disclosure.

FIG. 2A is a diagram illustrating biologically functional polynucleotides embodying features consistent with the principles of the present disclosure.

FIG. 2B is a diagram illustrating eGFP fluorescent signal in cells transfected with biologically functional polynucleotides encoding eGFP.

FIG. 3A is a diagram illustrating biologically functional polynucleotides embodying features consistent with the principles of the present disclosure.

FIG. 3B is a diagram illustrating eGFP fluorescent signal in cells transfected with biologically functional polynucleotides encoding eGFP.

FIG. 4A is a diagram illustrating biologically functional polynucleotides embodying features consistent with the principles of the present disclosure.

FIG. 4B is a diagram illustrating eGFP fluorescent signal in cells transfected with biologically functional polynucleotides encoding eGFP.

FIG. 5A is a diagram illustrating biologically functional polynucleotides embodying features consistent with the principles of the present disclosure.

FIG. 5B is a diagram illustrating eGFP fluorescent signal in cells transfected with biologically functional polynucleotides encoding eGFP.

FIG. 5C is a diagram illustrating eGFP fluorescent signal in cells transfected with biologically functional polynucleotides encoding eGFP.

FIG. 6A is a diagram illustrating biologically functional polynucleotides embodying features consistent with the principles of the present disclosure.

FIG. 6B is a diagram illustrating eGFP fluorescent signal in cells transfected with biologically functional polynucleotides encoding eGFP.

FIG. 7A is a diagram illustrating biologically functional polynucleotides embodying features consistent with the principles of the present disclosure.

FIG. 7B is a diagram illustrating eGFP fluorescent signal in cells transfected with biologically functional polynucleotides encoding eGFP.

FIG. 8A is a diagram illustrating biologically functional polynucleotides embodying features consistent with the principles of the present disclosure.

FIG. 8B is a diagram illustrating hemagglutinin (HA) expression (ELISA data) in cells transfected biologically functional polynucleotides encoding HA.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

### Exemplary Definitions

In accordance with long standing patent law convention, the words “a” and “an” when used in this application, including the claims, denote “one or more.”

The term “comprises” and grammatical equivalents thereof are used herein to mean that other components, steps, etc. are optionally present. For example, a biologically functional polynucleotide “comprising” components A, B, and C can contain only components A, B, and C, or can contain not only components A, B, and C, but also one or more other components.

The term “consisting of” and grammatical equivalents thereof are used herein to mean that other components, steps, etc. are not optionally present. For example, a biologically functional polynucleotide “consisting of” components A, B, and C can only contain components A, B, and C.

The terms “about” and “approximately,” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs), RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

The term “internal translation initiation site,” as used herein, describes an RNA element that allows for translation initiation in a cap-independent manner.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “treatment” or any grammatical variation thereof (e.g., treat, treating, and treatment etc.), as used herein, includes but is not limited to, alleviating a symptom of a disease or condition; and/or reducing, suppressing, inhibiting, lessening, ameliorating or affecting the progression, severity, and/or scope of a disease or condition. The term “effective amount,” as used herein, refers to an amount that is capable of treating or ameliorating a disease or condition or otherwise capable of producing an intended therapeutic effect.

The term “therapeutics” or any grammatical variation thereof, as used herein, includes, but is not limited to, drug therapies used for the treatment of patients having an existing disease or condition. Types of therapeutics that may be used for the treatment of existing diseases or conditions of a patient include, but are not limited to, mRNA-based therapies targeted at: cancer, infections, genetic disease, autoimmune, etc.

The term “preventatives” or any grammatical variation thereof, as used herein, includes, but is not limited to, drug therapies used for the purpose of preventing or ameliorating the effects of a disease or condition which a patient has not yet contracted. Types of preventatives that may be used to prevent or ameliorate the effects of a disease or condition include, but are not limited to, vaccines.

The term “carrier,” as used herein, refers to a diluent, adjuvant, excipient, or vehicle with which the compound is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for biological compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

As used herein, the term “DNA source” refers to any DNA fragment carrying a suitable polymerase promoter, internal translation initiation site, transgene, untranslated regions or linkers, enhancers, repressors and any other regulatory elements needed, including nucleotide sequences for hairpins, to create suitable mRNA transcripts. Alternatively, a DNA molecule that has been isolated free of total genomic DNA of a particular species may serve as a “DNA source.” Therefore, a DNA source obtained from a biological sample using one of the compositions disclosed herein may refer to one or more DNA sources that may or may not have been isolated away from, or purified free from, total genomic DNA of the particular species from which they are obtained. Included within the term “DNA source,” are DNA segments and smaller fragments of such segments, as well as recombinant vectors, including, for example, plasmids, cosmids, phage, viruses, PCR product, synthetic DNA, DNA ligation products and the like.

The term “effective amount,” as used herein, refers to an amount that is capable of treating or ameliorating a disease or condition or otherwise capable of producing an intended therapeutic effect.

The term “for instance” or “e.g.,” as used herein, is used merely by way of example, without limitation intended, and should not be construed as referring only those items explicitly enumerated in the specification.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise and may include the knowledge that the patient is ill as the result of a disease state that is treatable by one or more compound or pharmaceutical compositions such as those set forth herein.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, but are not limited to, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

The term “operably linked,” as used herein, refers to that the nucleic acid sequences being linked are typically contiguous, or substantially contiguous, and, where necessary, to join two or more structural elements of the vector or two or more protein coding regions. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In particular embodiments, the mammalian patient is preferably human.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, but are not limited to, acid addition salts formed with inorganic acids (e.g., hydrochloric acid, hydrobromic acid, sulfuric acid, phosphoric acid, nitric acid, and the like); and salts formed with organic acids including, but not limited to, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “1” isomeric form. However, residues in the “d” isomeric form may be substituted for any 1-amino acid residue provided the desired properties of the polypeptide are retained.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified and include any sugar or other derivative(s) or conjugate(s) added to the backbone amino acid chain.

The term “recombinant” indicates that the material (e.g., a polynucleotide or a polypeptide) has been artificially or synthetically (non-naturally) altered by human intervention. The alteration can be performed on the material within or removed from, its natural environment or state.

The term “subject,” as used herein, describes an organism, including mammals such as primates, to which treatment with the compositions according to the present invention can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, but not limited to, mice, rats, guinea pigs, rabbits, hamsters, and the like.

The term “substantially complementary,” when used to define nucleic acid sequences, means that a particular subject sequence, for example, an oligonucleotide sequence, is substantially complementary to all or a portion of the selected sequence, and thus will specifically self-anneal to a portion of an DNA or RNA having the selected sequence. As such, the number of acceptable unpaired nucleotides in a given sequence will depend on the total number of nucleotides in a given sequence and the point in which unpaired nucleotides cause stability issues within the sequence. For instance, the hairpin of the present invention comprises a “stem” and a “loop.” The “stem” of a sequence will comprise of no less than 4 paired nucleotides, but can be much greater than that. The number of unpaired nucleotides it may tolerate is the number of unpaired nucleotides to make the stem unstable, which, in a preferred embodiment, should be no more than 20% unpaired nucleotides. For instance, a stem having 10 paired nucleotides may tolerate 2 unpaired nucleotides. On the other hand, the “loop” of a sequence will comprise no less than 2 unpaired nucleotides. Those unpaired nucleotides may be complementary to each other, but because of steric folding issues, they cannot self-anneal with each other within the given hairpin and are thus ‘unpaired.’ However, nucleotides in a loop can be paired with other distant sequences within the molecule without disrupting the given hairpin. In many instances, it may be desirable for the sequences to be exact matches, i.e., be completely complementary to the sequence to which the oligonucleotide specifically self-anneals, and therefore have zero mismatches along the complementary stretch.

As used herein, the terms “treat,” “treating,” and “treatment” refer to the administration of one or more compounds (either alone or as contained in one or more pharmaceutical compositions) after the onset of clinical symptoms of a disease state so as to reduce, or eliminate any symptom, aspect or characteristic of the disease state. Such treating need not be absolute to be deemed medically useful. As such, the terms “treatment,” “treat,” “treated,” or “treating” may refer to therapy, or to the amelioration or the reduction, in the extent or severity of disease, of one or more symptom thereof, whether before or after its development afflicts a patient.

The term “vector,” as used herein, refers to any molecule/particle used as a vehicle to artificially carry an external nucleic acid sequence-usually DNA or RNA-into a target cell, where it can be replicated or expressed. A plasmid, PCR product, mRNA, virus, and/or cosmid are exemplary vectors.

As used herein, “an effective amount” would be understood by those of ordinary skill in the art to provide a therapeutic, prophylactic, or otherwise beneficial effect to a recipient patient.

The phrases “isolated” or “biologically pure” refer to material that is substantially, or essentially, free from components that normally accompany the material as it is found in its native state. Thus, isolated polynucleotides in accordance with the invention preferably do not contain materials normally associated with those polynucleotides in their natural, or in situ, environment.

“Link” or “join” refers to any method known in the art for functionally connecting one or more proteins, peptides, nucleic acids, or polynucleotides, including, but not limited to, recombinant fusion, covalent bonding, disulfide bonding, ionic bonding, hydrogen bonding, electrostatic bonding, and the like.

As used herein, the term “plasmid” refers to a genetic construct that is composed of genetic material (i.e., nucleic acids). Typically, a plasmid contains an origin of replication that is functional in bacterial host cells, e.g., Escherichia coli, and selectable markers for detecting bacterial host cells including the plasmid. Plasmids of the present invention may include one or more genetic elements as described herein arranged such that an inserted coding sequence can be transcribed and translated in a suitable expression cells. In addition, the plasmid may include one or more nucleic acid segments, genes, promoters, enhancers, activators, multiple cloning regions, or any combination thereof, including segments that are obtained from or derived from one or more natural and/or artificial sources.

Naturally, the present invention also encompasses nucleic acid segments that are complementary, essentially complementary, and/or substantially complementary to at least one or more of the specific nucleotide sequences specifically set forth herein. In a preferred embodiment, nucleic acid sequences that are “complementary” are those that are capable of base-pairing according to the standard Watson-Crick complementarity rules. However, RNA that can be folded and stabilized by non-canonical base pairing are also encompassed by this disclosure, including, but not limited to, Hoogsteen base pairs and Wobble base pairs. As used herein, the term “complementary sequences” means nucleic acid sequences that are substantially complementary, as may be assessed by the same nucleotide comparison set forth above, or as defined as being capable of hybridizing to one or more of the specific nucleic acid segments disclosed herein under relatively stringent conditions such as those described immediately above.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, but not limited to, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorigenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernable from the first label. The use of multiplexing assays, particularly in the context of genetic amplification/detection protocols are well-known to those of ordinary skill in the molecular genetic arts.

### Biologically Functional Polynucleotide

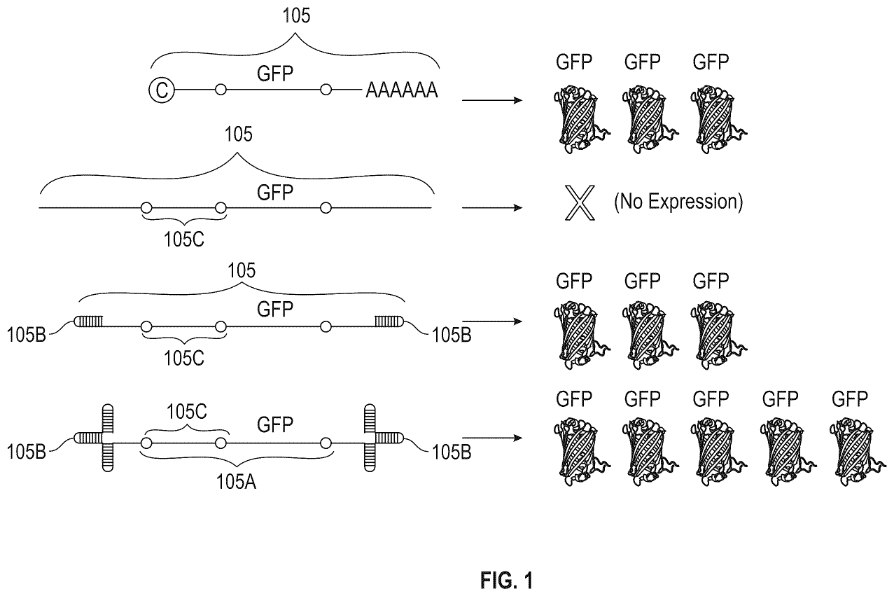

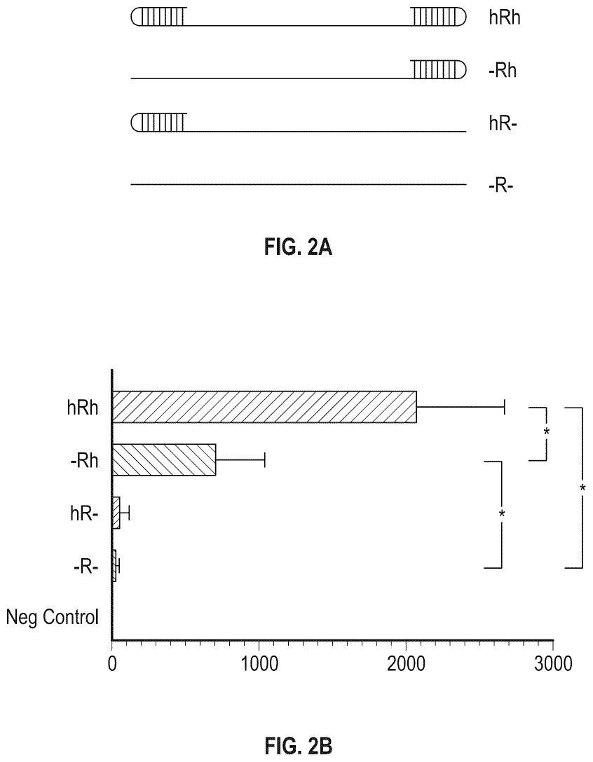

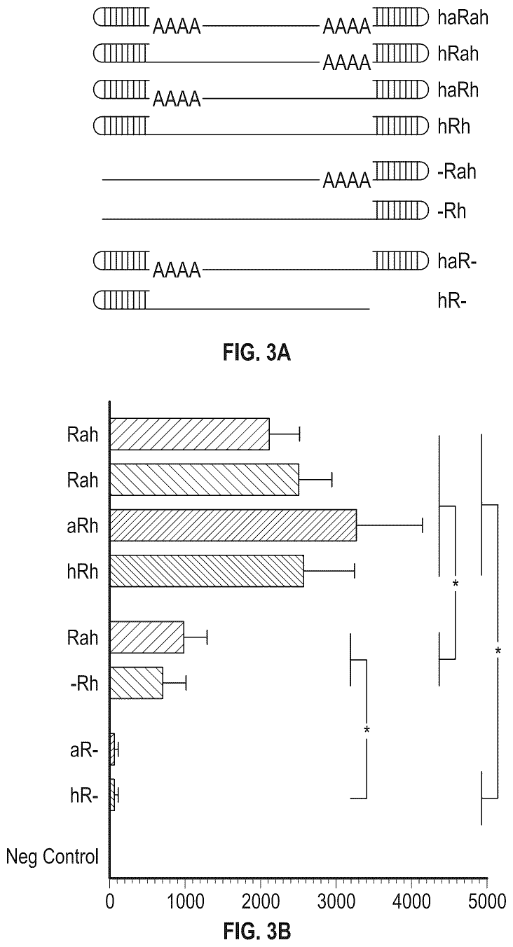

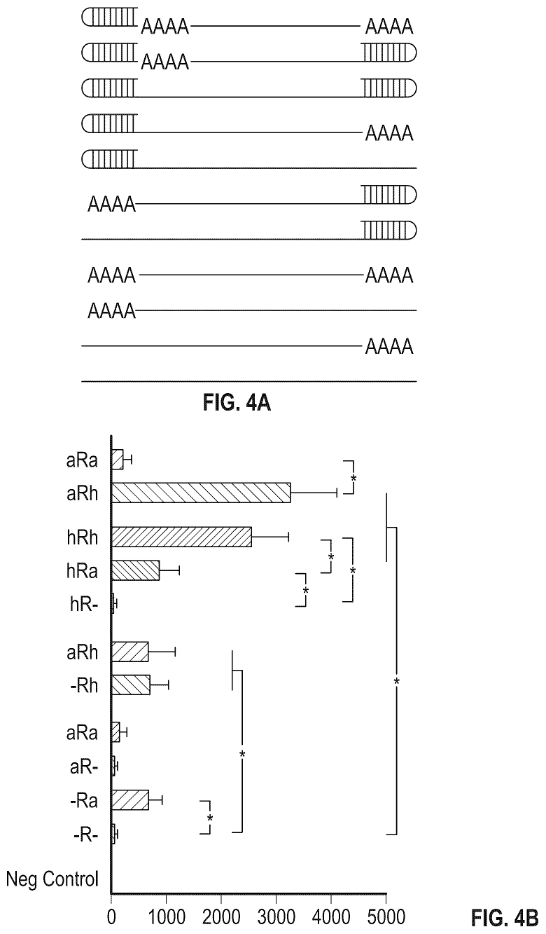

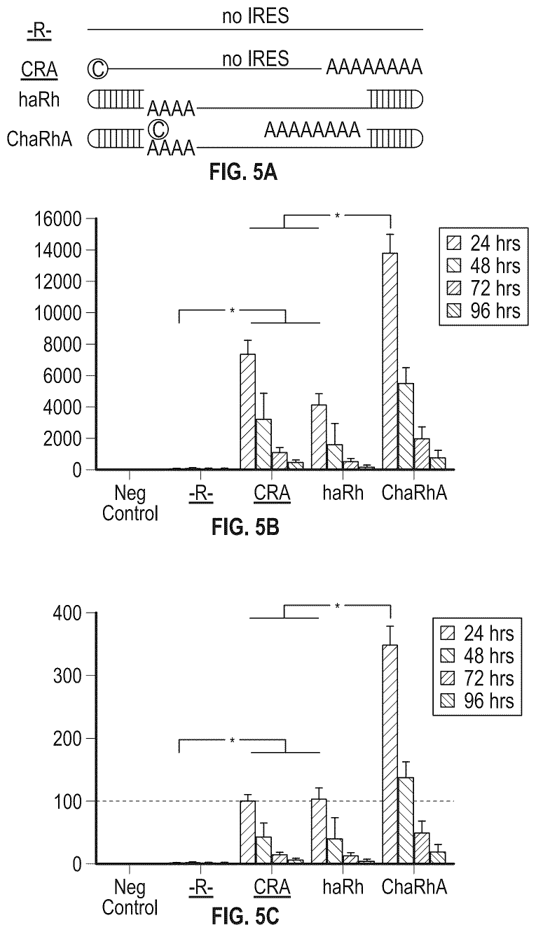

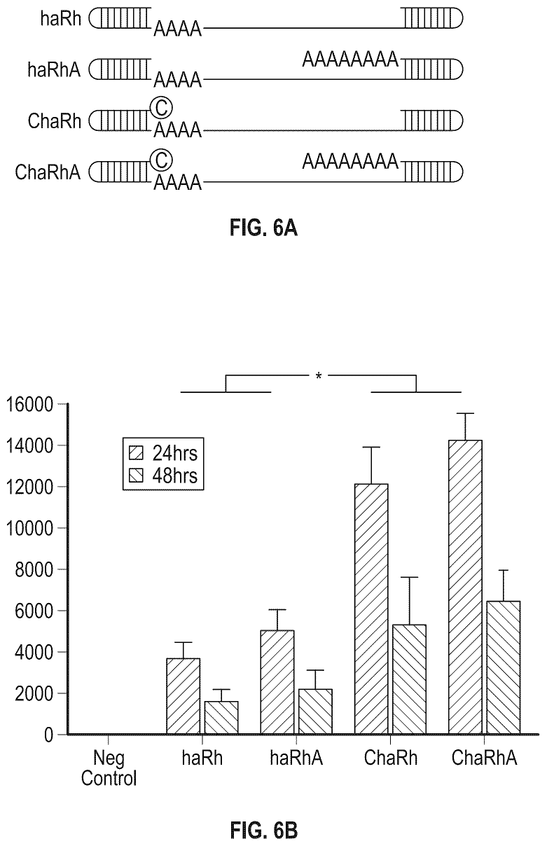

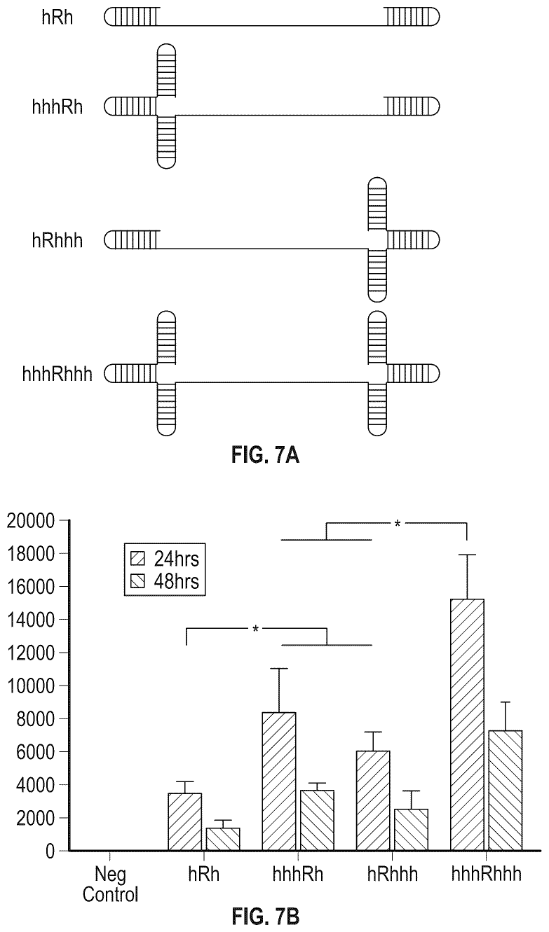

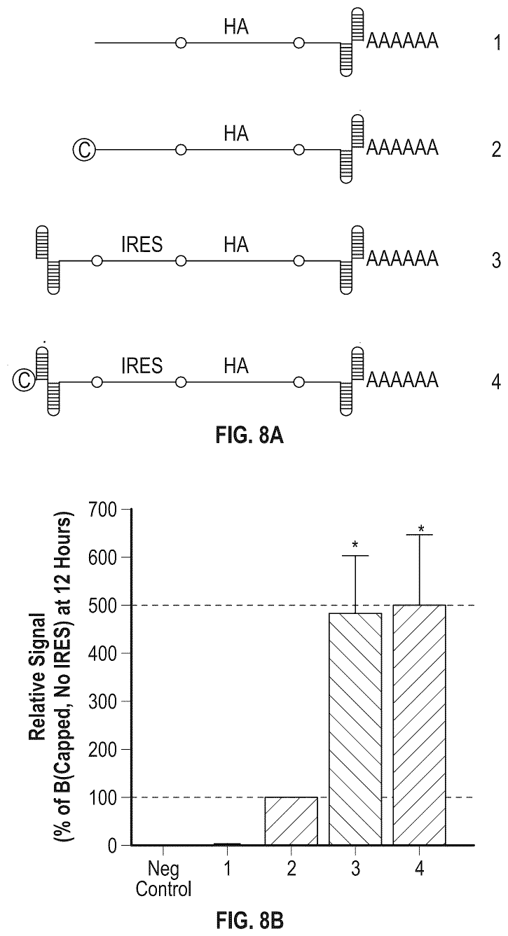

FIGS. 1-8B illustrate preferred embodiments of a biologically functional polynucleotide 105 to be used for therapeutic exogenous mRNA-based technologies as well as methods of producing the same. The biologically functional polynucleotide 105 is generally designed to be stable and functional without the need of a cap or a poly(A) tail. FIG. 1 depicts the design of several biologically functional polynucleotides 105 that can be used as therapeutic mRNA, including a biologically functional polynucleotides 105 lacking a cap and poly(A) tail. FIGS. 2A and 2B depict biologically functional polynucleotides 105 with corresponding eGFP fluorescent signal in transfected HEK293 cells 24 hours after electroporation. All four biologically functional polynucleotides 105 had identical sequences except in their terminal sequences that either formed or did not form stable hairpins 105B. FIGS. 3A and 3B depicts biologically functional polynucleotides 105 with corresponding eGFP fluorescent signal in transfected HEK293 cells 24 hours after electroporation. All biologically functional polynucleotides 105 had identical sequences except in their terminal sequences that either formed or did not form stable hairpins 105B and included or did not include short internal poly(A) sequences that flanked the 5′ and the 3′ terminal hairpins 105B. FIGS. 4A and 4B depict biologically functional polynucleotides 105 with corresponding eGFP fluorescent signal in transfected HEK293 cells 24 hours after electroporation. All vectors had identical sequences except where terminal hairpins 105B or short poly(A) sequences are indicated. FIGS. 5A-5C depict biologically functional polynucleotides 105 with corresponding eGFP fluorescent signal in transfected HEK293 cells 24, 48, 72, and 96 hours after electroporation. FIGS. 6A and 6B depict biologically functional polynucleotides 105 with corresponding eGFP fluorescent signal in transfected HEK293 cells 24 and 48 hours after electroporation. FIGS. 7A and 7B depict biologically functional polynucleotides 105 with corresponding eGFP fluorescent signal in transfected HEK293 cells 24 and 48 hours after electroporation. FIGS. 8A and 8B depict biologically functional polynucleotides 105 with corresponding hemagglutinin (HA) expression (ELISA data) in transfected MDCK cells after 12 hours. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

In a preferred embodiment, the biologically functional polynucleotide 105 is mRNA that consists of a target mRNA designed to form hairpins 105B at both the 5′ end and 3′ end, wherein said hairpins 105B comprise a stem structure and loop structure. The stem is contiguous with the rest of mRNA transcript or chemically synthesized mRNA molecule 105A and preferably comprises at least 80% paired nucleotides and can tolerate up to two unpaired nucleotides at the mRNA end. The loop is connected to the stem and preferably comprises at least 2 unpaired nucleotides in a given hairpin. The hairpins 105B of said biologically functional polynucleotide 105 are preferably formed by the pairing of no less than 4 complementary base pairs, resulting in a segment of double stranded mRNA at the 5′ and 3′ ends. Each hairpin 105B contains at least one loop; however, other preferred embodiments of the biologically functional polynucleotide 105 may comprise a cap, tail, or both a cap and a tail without departing from the inventive subject matter described herein.

In a preferred embodiment, the following (hairpin) sequences at the 5′ and 3′ end are preferred:

Double Hairpin Structures
5′ end
GGGCCGUCCGGGCAAUUGCCCGGACGGCCCCCCGGC
AGCCCGCAAUUGCGGGCUGCCGGG>>>>>
3′ end
>>>>>GCGCGCCAGGGCGCCAAUUGGCGCCCUGGCGC
GCCCGCGGGUGGGCGCCAAUUGGCGCCCACCCGCGG
Triple Hairpin Structures
5′ end
GGGAGACCCGAGCUCGGAUCCGAGCUCGGAUCCGAGC
UCGGGUCUCCCGCUGCCUGUCCGGGCAUAUGCCCGGA
CAGGCAGCGACGCUGGCUGGACGCUUAAGCGUCCAGC
CAGCGUC>>>>>
3′ end
>>>>>CGGACGACGUCGCUCAGGUAUACCUGAGCGAC
GUCGUCCGACCGGUGCUCACGUGCGGCUGCAGCCGCA
CGUGAGCACCGGUGGGUCGAGGCUGAUCGGCGAGCUC
GGUUAACCGAGCUCGCCGAUCAGCCUCGACCC

However, the effect of these terminal hairpin structures on mRNA functionality is largely sequence-independent and depends primarily on the stability of the hairpins formed. In another preferred embodiment, the stable hairpins comprise a range of 20-40 nucleotides and possess a higher percentage of GC in the stem. However, one with skill in the art will understand that stable hairpins having a different number of nucleotides and/or comprising a different combination of nucleotides may also be used to produce the biologically functional polynucleotides and does not depart from the inventive subject matter described herein.

The target mRNA preferably comprises an mRNA transcript or chemically synthesized mRNA molecule 105 that includes an internal translation initiation site 105C incorporated 105C upstream of a delivered gene sequence. In a preferred embodiment, the internal translation initiation site 105C is internal ribosome entry site (IRES) and is incorporated into said mRNA transcript 105 (or said chemically synthesized mRNA molecule) within the 5′ untranslated region (5′ UTR) of said mRNA transcript 105 (or chemically synthesized mRNA molecule). The hairpins 105B of the target mRNA 105 are contiguous with the internal translation initiation site 105C the delivered gene sequence, and untranslated regions of said mRNA transcript or chemically synthesized mRNA molecule 105A at one or both of the 5′ end and 3′ end. Hairpins 105B of the biologically functional polynucleotide 105 include, but are not limited to, single hairpins, double hairpins, and triple hairpins, wherein said double hairpins and triple hairpins have zero or a minimal number of unpaired nucleotides separating each individual hairpin. Therefore, embodiments of the biologically functional polynucleotide 105 may comprise a target mRNA having a plurality of different types of hairpins 105B without departing from the inventive subject matter described herein. For instance, a biologically functional polynucleotide 105 may comprise an mRNA having a double hairpin operably linked to the target mRNA at the 5′ untranslated region and a double hairpin operably linked to the target mRNA at the 3′ untranslated region. For instance, a target mRNA 105 may have a single hairpin at the 5′ end and a triple hairpin at the 3′ end. Further, the hairpin 105B at the 5′ end is operably linked to the internal translation initiation site 105C by a linker nucleotide sequence that can vary in sequence and number whereas the hairpin 105B at the 3′ untranslated region is operably linked to the open reading frame (ORF) of the delivered gene by another linker nucleotide sequence that may also vary in sequence and number. Additionally, the linker nucleotide sequences to which the hairpins 105B are operably linked may differ in sequence and number.

The DNA sequence used to create the target mRNA is first inserted into a DNA source (plasmid DNA, genomic DNA, synthetic DNA, PCR product, etc.) to create a DNA template, wherein said DNA template is configured to encode the RNA transcripts containing the target mRNA 105 having the internal translation initiation site 105C incorporated upstream of a transgene and contiguous with hairpins 105B at both ends. In vitro transcription is then accomplished using T7 RNA polymerase; however other RNA polymerases, such as SP6, T3, and others, may be used without departing from the inventive subject matter described herein. This results in the target mRNA vector 105 harboring the internal translation initiation site 105C upstream of a transgene. Further, because the DNA template is configured to produce an mRNA transcript with an internal translation initiation site 105C incorporated therein and having hairpins 105B contiguous with both the 5′ and 3′ ends, no cap or poly(A) tail must be added in subsequent steps, allowing the creation of stable and functional therapeutic exogenous mRNA in a single step. Natural and modified ribonucleotides (or their combinations) may be used for the synthesis of the target mRNA 105. Alternatively, the terminal hairpin 105B can be added to mRNA at the 5′ end only. In this case, a poly(A) tail may be needed at 3′ end for mRNA protection and proper functionality. Likewise, the terminal hairpin 105B can be added to target mRNA 105 at the 3′ end only. In this case, a cap may be needed at 5′ end for mRNA protection and proper functionality.

Alternatively, the target mRNA 105 may be chemically synthesized without a DNA template to produce therapeutic exogenous mRNA in a single step. Currently, chemically produced RNA (i.e. RNA produced in vitro without the use of a DNA template) is almost always synthesized using automated solid-phase methods. Other methods include solid-phase synthesis of RNA combined with chemical ligation to join multiple chemically synthesized strands of RNA to make mRNA.

### Therapeutic and Preventative Uses

Another aspect of the invention pertains to uses of the target mRNA 105 to encourage efficient transfection of cells, tissues, and/or organs of interest, and/or for use in therapeutics and preventatives, such as gene-based therapies and vaccines, respectively.

In one embodiment, the present invention provides a method for generating therapeutic or experimental mRNA for delivering into cells, tissues, and/or organs of interest, comprising introducing into a cell, a composition comprising an effective amount of a single chained polynucleotide comprising the target mRNA 105 of present invention, which may include at least one regulatory element. In particular embodiments, the mRNA regulatory elements of the present invention may be used to affect control of protein expression from one or more mRNAs encoding one or more gene products, therapeutic agents, proteins, or such like, in suitable host cells.

In addition, the present invention provides a method for treatment of a disease, wherein the method comprises administering, to a subject in need of such therapeutic and/or vaccination, an effective amount of a composition comprising one or more polynucleotide sequences encoding a selected protein of interest operably positioned with one or more of the mRNA regulatory elements disclosed herein, such that the regulatory element is able to affect, alter, reduce, increase, or otherwise control translation of the encoded protein(s) from the mRNA for which the regulatory element is affecting translation.

The invention also provides for the use of a composition disclosed herein in the manufacture of a medicament for treating, preventing or ameliorating the symptoms of a disease, disorder, dysfunction, injury or trauma, including, but not limited to, the treatment, prevention, and/or prophylaxis of a disease, disorder or dysfunction, and/or the amelioration of one or more symptoms of such a disease, disorder or dysfunction. Exemplary conditions for which the biologically functional polynucleotide 105 may find particular utility include, but are not limited to, viral, bacterial or other pathogens infection, cancer, diabetes, allergy, autoimmune disease/disorder, kidney disease, cardiovascular disease, pancreatic disease, intestinal disease, liver disease, neurological disease, neuromuscular disorder, neuromotor deficit, neuroskeletal impairment, neurological disability, neurosensory dysfunction, stroke, al-antitrypsin (AAT) deficiency, Batten's disease, ischemia, an eating disorder, Alzheimer's disease, Huntington's disease, Parkinson's disease, skeletal disease and pulmonary disease.

The invention also provides a method for treating or ameliorating the symptoms of such a disease, injury, disorder, or dysfunction in a mammal. Such methods generally involve at least the step of administering to a mammal in need thereof, one or more biologically functional polynucleotide 105 of the present invention, in an amount and for a time sufficient to treat or ameliorate the symptoms of such a disease, injury, disorder, or dysfunction in the mammal.

Such treatment regimens are particularly contemplated in human therapy, via administration of one or more compositions either intramuscularly, intravenously, subcutaneously, intrathecally, intraperitoneally, or by direct injection into an organ or a tissue of the subject under care.

The invention also provides a method for providing to a mammal in need thereof, a therapeutically-effective amount of the biologically functional polynucleotide 105 of the present invention, in an amount, and for a time effective to provide the patient with a therapeutically-effective amount of the desired therapeutic agent(s) encoded by one or more nucleic acid segments comprised within the biologically functional polynucleotide 105. Preferably, the therapeutic agent is mRNA, but other embodiments of the biologically functional polynucleotide 105 may be selected from the group consisting of a ribozyme, peptide nucleic acid, siRNA, RNAi, antisense oligonucleotide, and an antisense polynucleotide.

### Pharmaceutical Compositions

The present invention also provides therapeutic or pharmaceutical compositions comprising the active ingredient in a form that can be combined with a therapeutically or pharmaceutically acceptable carrier. The genetic constructs of the present invention may be prepared in a variety of compositions and may also be formulated in appropriate pharmaceutical vehicles for administration to human or animal subjects.

The biologically functional polynucleotides 105 of the present invention provide new and useful methods for the regulation of protein translation in suitable mammalian cells and offer new opportunities for the expression of one or more selected genes of interest in said mammalian cells. The biologically functional polynucleotides 105 of the present invention also provide compositions comprising one or more of the disclosed biologically functional polynucleotides 105.

As described herein, the compositions of the present invention may further comprise a pharmaceutical excipient, buffer, transfection reagent, nanoparticle, nanolipoprotein particle, lipid nanoparticle (NLPs), or diluent, and may be formulated for administration to an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions which result in diseases or disorders such as viral, bacterial or other pathogens infection, cancer, tumors, or other malignant growths, neurological deficit dysfunction, allergy, autoimmune diseases/disorders, articular diseases, cardiac or pulmonary diseases, ischemia, stroke, cerebrovascular accidents, transient ischemic attacks (TIA); diabetes and/or other diseases of the pancreas; cardiocirculatory disease or dysfunction (including, e.g., hypotension, hypertension, atherosclerosis, hypercholesterolemia, vascular damage or disease; neural diseases (including, e.g., Alzheimer's, Huntington's, Tay-Sach's and Parkinson's disease, memory loss, trauma, motor impairment, neuropathy, and related disorders); biliary, renal or hepatic disease or dysfunction; musculoskeletal or neuromuscular diseases (including, e.g., arthritis, palsy, cystic fibrosis (CF), amyotrophic lateral sclerosis (ALS), multiple sclerosis (MS), muscular dystrophy (MD), and such like).

In some preferred embodiments, target mRNA 105 having at least one hairpin 105B may be formulated with one or more pharmaceutically acceptable solutions for administration to a cell or an animal, either alone or in combination with one or more other modalities of therapy, and in particular, for therapy of human cells, tissues, and diseases affecting man. This includes embodiments of the biologically functional compound also containing at least one of a cap and poly(A) tail. If desired, additional nucleic acid segments may be operably linked to the target mRNA vector 105, and the products thereof may then be administered to an animal (either alone, or in combination with one or more other agents as well, such as, e.g., proteins or polypeptides or various pharmaceutically-active agents, therapeutic polypeptides, biologically active fragments, or variants thereof). In fact, there is virtually no limit to other components that may also be included, given that the additional agents do not cause a significant adverse effect upon contact with the target cells or host tissues and/or prevent the target mRNA 105 from being expressed within said animal to which said biologically functional polynucleotide 105 was administered. Therefore, compositions containing said biologically functional polynucleotide 105 may be delivered along with various other agents as required for a particular instance. Such compositions may be purified from host cells or other biological sources, or alternatively may be chemically synthesized as described herein.

Formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intra-articular, intramuscular administration and formulation. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the disclosed biologically functional polynucleotide 105 in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. The methods of administration may also include those modalities as described in U.S. Pat. Nos. 5,543,158, 5,641,515 and/or 5,399,363 (each of which is specifically incorporated herein in its entirety by express reference thereto). Solutions of the active compounds as freebase or pharmacologically acceptable salts may be prepared in sterile water and may also suitably mixed with one or more surfactants, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms of the compositions containing the disclosed biologically functional polynucleotide 105 that are suitable for injectable use include sterile aqueous solutions or dispersions and sterile powders for the extemporaneous preparation of sterile injectable solutions or dispersions (U.S. Pat. No. 5,466,468, specifically incorporated herein in its entirety by express reference thereto). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The compositions of the present invention can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection. In preferred embodiments, the composition is administered via intranasal, pulmonary, or oral route.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the biologically functional polynucleotides 105 in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Compositions containing the disclosed biologically functional polynucleotides 105 may also be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

The amount of a composition containing the disclosed biologically functional polynucleotides 105 and time need to administer said composition will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the composition containing the disclosed biologically functional polynucleotides 105, either over a relatively short, or a relatively prolonged period of time, as may be determined by the medical practitioner overseeing the administration of such compositions.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

The ability of uncapped and non-adenylated RNA vectors with unstructured UTRs to express a fluorescent reporter (eGFP) from a EMCV IRES to that of target mRNA 105 of the same length, but with a stable terminal hairpin 105B on one or more ends, is presented herein in the form of a biologically functional polynucleotide having transcripts or chemically synthesized mRNA molecules operably linked to hairpins 105B at one or more ends. The use of an internal translation initiation sequence allowed for the generation of target mRNA 105 without a 5′ cap and a poly(A) tail. The lack of a 5′cap and 3′poly(A) tail leaves these RNAs very susceptible to degradation from exonucleases, which significantly decreases their expression efficiency. Including stable terminal hairpins 105B at each end of the transcript or chemically synthesized mRNA molecule 105A lacking a cap and poly(A) tail may increase protein translation by reducing their sensitivity to exonuclease activity and/or by influencing regulatory mechanisms of protein translation. The effect on the efficiency of eGFP expression 24 hours after vector delivery into HEK293 cells by electroporation (FIG. 2) when incorporating stable secondary RNA hairpin structure(s) on either or both ends of mRNA was studied.

The results were compared to eGFP expression from a control vector (—R—) that contained an IRES and had the same length 5′ and 3′ UTR but lacked stable terminal hairpin structures. As illustrated in FIG. 2, transfection with the control vector having no hairpins 105B resulted in a very low eGFP expression. eGFP expression increased slightly if the transcript or chemically synthesized mRNA molecule 105 contained a 5′ hairpin (hR-), but the presence of a hairpin 105B at the 3′ end (-Rh) had a significantly greater positive effect on eGFP expression. Delivery of a transcript or chemically synthesized mRNA molecule 105 having both 5′ and 3′ hairpins (hRh) resulted in the greatest eGFP expression. These findings indicated that the addition of terminal secondary structures improved EMCV IRES-initiated translation in target mRNAs105 lacking a cap and a poly(A) sequence.

### Example 2

Internal polyadenosine sequences can serve as an additional point of entry for the poly(A) binding protein (PABP) and play an important role in regulating gene expression, especially in MSCV IRES-driven vectors that show less dependence on the 3′ poly(A) terminal tract for translation initiation. To determine whether such internal poly(A) sequences would improve translational efficiency in target mRNAs105 having one or more terminal hairpins, relatively short (40 bp) poly(A) stretches were inserted immediately downstream of the 5′ terminal hairpin or upstream of the 3′ terminal hairpin, and the eGFP expression was subsequently assessed. All vectors had an identical IRES-eGFP internal transcription cassette and differed only by the presence or absence of the terminal hairpins and poly(A) segments. HEK293 cells were electroporated with these target mRNA and eGFP signal was measured 24 hours later.

As illustrated in FIG. 3, the presence of poly(A) sequences downstream of the 5′ hairpin had little to no effect on eGFP expression if the 3′ end was unstructured (i.e. lacked a hairpin; haR- and hR-). Similar to Example 1, target mRNAs105 having a hairpin 105B at the 3′ end expressed more eGFP than target mRNAs105 having only a hairpin 105B at the 5′ end, but the addition of a poly(A) stretch immediately upstream of the 3′ hairpin (-Rh vs -Rah) had minimal effect on expression. Compared to target mRNAs105 having single hairpins, eGFP expression was greater in all vectors that had both 5′ and 3′ hairpins. The addition of poly(A) sequences to target mRNAs105 with terminal hairpins had minimal impact on eGFP expression.

### Example 3

Next, the effect of replacing terminal hairpins with short poly(A) stretches was investigated to determine if this would increase eGFP expression. The addition of a longer poly(A) tail at the 3′ end of mRNA increases the protein expression of capped messengers by stabilizing RNA and activating translation regulatory mechanisms. Whether this is also true with short poly(A) stretches when the transcript or chemically synthesized mRNA molecule 105 initiate translation from an internal IRES is not well studied.

The 5′ and 3′ terminal hairpins were replaced with short poly(A) stretches, and the translation efficiency of these target mRNAs105 in HEK293 cells was determined. Cells were electroporated with the appropriate vector and eGFP expression was measured 24 hours after vector delivery. As illustrated in FIG. 4, the addition of a short terminal 3′ poly(A) sequence modestly increased eGFP expression in target mRNAs105 that lacked a 3′ terminal hairpin (—R— vs -Ra, aR vs aRa, and hR- vs hRa), whereas adding a short terminal 5′ poly(A) sequence in target mRNAs105 that lacked a 5′ terminal hairpin had no significant effect on eGFP expression (aR- vs -R-, aRa vs -Ra, aRh vs -Rh). The positive effect of hairpins 105B on eGFP expression was much greater than the effect of short terminal poly(A) sequences. Target mRNAs105 having hairpins 105B at both ends of the transcript or chemically synthesized mRNA molecule 105 had the highest eGFP expression compared to vectors with other terminal end configurations.

### Example 4

Transcripts or chemically synthesized mRNA molecules operably linked to terminal hairpins were able to support higher levels of eGFP expression. Determining whether capping and posttranslational polyadenylation with a longer poly(A) tail affected translational efficiency in target mRNAs105 having both 5′ and 3′ hairpins was investigated. As illustrated in FIG. 4, a biologically functional polynucleotide 105 having transcripts or chemically synthesized mRNA molecules operably linked to bilateral terminal hairpins coupled with an internal poly(A) sequence downstream of the 5′ terminal hairpin (haRh) vector had the best expression efficiency. Therefore, it was selected as the preferred biologically functional polynucleotide 105 for these experiments. A vector with no IRES sequence, but with the same eGFP ORF, was used for comparison (—R—). The length of the 5′ and 3′ UTRs of this vector were comparable to those in the vector haRh, but were intentionally designed to not form stable secondary structures (i.e. not form terminal hairpins). These two (control) vectors (—R— and haRh) were then capped with 3′-O-Me-m7G (5′) ppp (5′) G RNA Cap Structure Analog (also known as Anti-Reverse Cap Analog (ARCA)) and poly-adenylated using E. coli Poly (A) Polymerase resulting in a tail length of greater than 200 bases, yielding two additional vectors—(ChaRhA) and (CRA).

eGFP levels in targeted cells were measured between 24 to 96 hours after vector delivery (FIGS. 5B and 5C) in order to assess the stability of protein expression. Cell transfection was achieved by electroporation. The control vector —R—, which lacked a 5′ cap and long terminal poly(A), expressed barely detectable eGFP. Vectors CRA (which initiated translation using a 5′cap and was poly-adenylated) and haRh (which initiated translation through an IRES and lacked a cap and poly(A) tail) had comparable eGFP expression when used in equimolar concentrations (FIG. 5C). The vector that combined bilateral hairpins, a 5′ cap, a long poly(A) tail, and a MSCV IRES (ChaRhA) resulted in the highest eGFP level at all-time points. The fluorescent signal decayed at a consistent rate in all cells 24 to 96 hours after electroporation.

To determine which modification was primarily responsible for the increased eGFP expression from the ChaRhA vector (the Cap or the long Poly (A) tail, two more biologically functional polynucleotides 105 were generated: ChaRh which was capped but lacked a long poly(A) tail and haRhA which was not capped but had a long poly(A) tail (FIG. 6A). Although both modifications increased eGFP expression, as illustrated in FIG. 6B, the addition of the cap (ChaRh) had a greater effect than the addition of the poly(A) tail (haRhA). The effect of adding both a cap and a poly(A) tail was additive. Similar to the findings in FIG. 5, there was no difference between vectors in the rate of eGFP signal decay over time.

### Example 5

It is possible that the presence of an even more complex secondary structures in close proximity to either end of the RNA may protect this end from exonucleases by physically limiting access of the exonucleases to the terminus.

To test the possibility that the presence of even more complex secondary structures in close proximity to either end of the RNA may protect this end from exonucleases by physically limiting access of the exonucleases to the terminus, it was investigated whether a triple terminal hairpin structure in the delivered RNA improved protein expression above that of a single hairpin. Since the inclusion of poly(A) stretches in previous experiments had a minimal effect on eGFP expression, three new biologically functional polynucleotides 105 having no internal poly(A) sequences were constructed and compared to a target mRNAs105 harboring single hairpins on each end (hRh) (FIG. 7A). In one vector we replaced a single 5′ hairpin with a triple hairpin structure (hhhRh). The individual hairpins 105B were linked with no unpaired linkers. The second vector was constructed by replacing a single 3′ hairpin in the hRh vector with a 3′ triple hairpin structure using the same strategy (hRhhh). The last vector had triple hairpin structures on both ends (hhhRhhh).

To assess the stability of protein expression, eGFP levels were measured in HEK293 cells transfected with each vector 24 and 48 hours after vector delivery by electroporation (FIG. 7B). All vectors with triple hairpin structures demonstrated enhanced eGFP expression compared to the single hairpin vector (hRh). The vector that combined triple hairpin structures at both the 5′ and 3′ end (hhhRhhh) resulted in the highest eGFP level at both time points. Of specific note, the fluorescent signal at 48-hour post-electroporation was about 42-43% of the value seen at 24-hours for all vectors except for the one with triple hairpins at both ends (hhhRhhh) where fluorescence was 48%. The decrease in fluorescent decay rate is likely explained by improved vector stability in cells.

### Example 6

To assess what exclusive effect the use of the 5′ double hairpin and IRES might have on expression of membrane proteins, mRNA vectors encoding an Influenza hemagglutinin (HA) in place of eGFP were tested. Accordingly, as illustrated in FIG. 8A, four more biologically functional polynucleotides 105, differing only at the 5′ mRNA end, were generated: 1) mRNA vector with no cap, no IRES, 2) mRNA vector with a cap (C, ARCA) and no IRES, 3) mRNA vector with no cap, with the 5′ double hairpin and IRES, and 4) mRNA vector with a cap (C, ARCA), the 5′ double hairpin and IRES. (n=2, *P<0.05). All four vectors had the 3′ double hairpin and a poly(A) tail. FIG. 8B illustrates HA expression (ELISA data) when equal amounts of mRNA were used in MDCK cells in 12 hours after transfection with TransIT®-mRNA reagent. As seen in FIG. 8B, the use of the 5′ double hairpins and IRES increased HA expression about 5 times compared to the vector with a 5′ cap and no IRES. Further, adding a 5′ cap to the mRNA vector with the 5′ double hairpin and IRES had little effect on the total HA expression.

### Discussion

Here we show that mRNA designed to form hairpin secondary structures at both the 5′ and 3′ ends maintains a high level of reporter expression in eukaryotic cells, even in the absence of a 5′ cap and 3′ polyadenylated tail, as long as an EMCV IRES is included in its 5′ UTR. Equimolar levels of EMCV IRES-containing mRNA showed the same level of protein expression as conventionally constructed (non-IRES-containing) mRNA that contained a 5′ cap and a 3′ poly-(A) tail. The presence of internal rather than terminal poly(A) stretches did not significantly influence protein expression. Combining post-transcriptional capping and poly-adenylation with terminal hairpins resulted in greater translation efficiency than either strategy alone. Using a triple hairpin structure instead of a single hairpin further increased protein expression, outperforming capped and poly-adenylated vectors without IRES.

In general, therapeutic mRNA is produced in three steps: 1) in vitro mRNA synthesis from a DNA template, 2) the addition of a modified guanosine cap on the 5′ end of the mRNA, and 3) the addition of a poly(A) tail on the 3′ end of mRNA. mRNA may be capped during transcription by including the cap analog in the nucleotide mix during synthesis or the cap can be added after the mRNA is completely transcribed. Regardless of the method used, it always results in a fraction of mRNA that is uncapped which renders it translationally inactive. In terms of the poly(A) tail, relatively short poly(A) tails can be directly added to the end of the mRNA during transcription by including the sequence into the DNA template. Alternatively, longer poly(A) tails that result in more stable mRNA can be added after in vitro transcription using recombinant poly(A) polymerase.

The question is whether mRNA can be synthesized without a cap and poly(A) tail and still function when introduced into cells. For in vitro mRNA transcripts to function without a 5′ cap would require an alternative way to initiate protein synthesis. One way to do this is to include an internal ribosome entry site (IRES) in the 5′ region to initiate translation. Cells use IRESs to increase translation of certain proteins during mitosis and programmed cell death. IRESs are often used by viruses to ensure that viral translation is active when host translation is inhibited. Although plus-strand RNA virus genomes that utilize internal ribosome entry sites (IRESs) to promote cap-independent translation are influenced by poly(A) binding proteins (PABP) and poly(A) status, transcription from other IRESs show much less dependence on the polyadenylation status. The EMCV IRES used in the vectors described here does rely on the conventional set of eukaryotic initiation factors (except eIF4E and intact eIF4G), but it does not require PABP or 5′-3′ communication with the poly(A) tail in vitro at least during the first-round of initiation. Thus, using an IRES rather than a 5′ cap to initiate protein synthesis allows for removal of the poly(A) tail without significantly impairing protein synthesis.

It should be possible, therefore, to produce uncapped and non-adenylated mRNA with an open reading frame downstream of an IRES that efficiently translates protein(s). This approach has not been used in either molecular biology applications or in vaccine or therapeutic drug production, however, because the un-capped 5′ and non-adenylated 3′ end are extremely sensitive to exonucleases degradation reducing mRNA stability.

One way to protect exogenously generated uncapped and non-adenylated mRNA from exonuclease degradation is to construct circular RNA. EMCV IRES driven reporter RNA vectors can be engineered to form circular RNAs lacking both a cap and poly(A). Such circular RNAs do not have free ends that are vulnerable to exonucleases and thus showed an increased stability that resulted in extended duration of protein expression. However, circular RNAs lack flexibility due to their rigid secondary structure and transfection of cells with exogenous circular RNA results in the activation of antiviral gene products such as OAS, PKR, and RIG-I which can initiate the cellular response against circular RNA.

An alternative method for protecting the terminal ends of mRNA lacking a cap and poly(A) tail is to include nucleotide hairpins at the 5′ and 3′ ends. A nucleotide hairpin is a pairing of complementary base pairs that is an essential secondary structure of RNA. It can guide RNA folding, determine interactions with ribozymes, protect mRNA from degradation, serve as a recognition motif for RNA binding proteins or act as a substrate for enzymatic reactions. It has been shown that a 5′-terminal stem-loop structure can stabilize mRNA in different bacteria probably by preventing RNase E from interacting with the 5′ end of the message. Of note, it appears that the location of this stem-loop at, or very near, the 5′ and 3′ terminus is crucial to its stabilizing effect, whereas the sequence of this hairpin and its position relative to the ribosome binding site appears to have little effect. Up to two unpaired nucleotides upstream of the 5′ hairpin are tolerated without any reduction in mRNA stability, but the addition of 10-15 unpaired nucleotides of random sequence is as destabilizing as deletion of the 5′ hairpin. A strong Shine-Dalgarno sequence near the 5′ end of the message in E. coli can recruit ribosomes and stabilize the message by blocking access of nucleases to degradative signals present in the naked mRNA.

A role for terminal hairpin structures in eukaryotes has not been widely studied because such structures appear to be uncommon in metazoans. At the 5′ end, a terminal hairpin can interfere with cap-induced processes, and at the 3′ end, the majority of mRNAs are flanked by a polyadenylation signal followed by 10-30 downstream nucleotides and a poly(A) tail which makes the formation of a terminal hairpin unlikely. Non-polyadenylated mRNAs are rare in eukaryotes.

We hypothesized that we could protect both ends of the exogenous mRNA vector by adding stable hairpins 105B during in vitro synthesis; this could stabilize the molecule and protect the mRNA from exonuclease degradation when delivered to target cells. However, this could be possible only with mRNA that does not depend on their ends to function. Including an EMCV IRES in the 5′ end to initiate translation would allow us to generate in vitro mRNA transcripts in a single step, bypassing the costly and time-consuming 5′ capping and 3′ poly(A) addition.

Using this strategy, we generated an effective mRNA transcript that was the equal of conventionally constructed mRNA that contained a 5′ Cap and a 3′ poly(A) tail. The use of triple hairpins at each end led to the greatest increase in reporter expression of any vector tested (i.e. conventional construction, hairpins 105B with IRES, or hairpins 105B with IRES with cap and poly(A) tail). Such mRNA structures are uncommon in eukaryotic cell, but they can be easily synthetized in vitro in a single step and then delivered as drugs or vaccines. In these experiments, we used the EMCV IRES, but other IRESs may potentially be used as well. However, each IRES may function differently depending on the vector, so results would need to be validated experimentally.

These results demonstrate that a 5′ cap and a 3′ poly(A) tail are not always required for the successful expression of exogenously generated mRNA in eukaryotic cells. The inclusion of a 5′ IRES is sufficient to initiate translation of the encoded protein. The inclusion of hairpins 105B (single or triple) at each end protects the mRNA against degradation by exonucleases. This provides a potential method for rapidly generating exogenous mRNA using a single step, thus saving time and reducing cost.

### REFERENCES

The following references, to the extent that they provide exemplary procedural or other details supplementary to those set forth herein, are specifically incorporated herein in their entirety by express reference thereto:
1. Teijaro, J. R., and Farber, D. L. (2021). COVID-19 vaccines: modes of immune activation and future challenges. Nat Rev Immunol 21, 195-197. 10.1038/s41577-021-00526-x.
2. Wadhwa, A., Aljabbari, A., Lokras, A., Foged, C., and Thakur, A. (2020). Opportunities and Challenges in the Delivery of mRNA-based Vaccines. Pharmaceutics 12. 10.3390/pharmaceutics12020102.
3. Ramanathan, A., Robb, G. B., and Chan, S. H. (2016). mRNA capping: biological functions and applications. Nucleic Acids Res 44, 7511-7526. 10.1093/nar/gkw551.
4. Ghosh, A., and Lima, C. D. (2010). Enzymology of RNA cap synthesis. Wiley Interdiscip Rev RNA 1, 152-172. 10.1002/wrna.19.
5. Shuman, S. (2001). Structure, mechanism, and evolution of the mRNA capping apparatus. Prog Nucleic Acid Res Mol Biol 66, 1-40. 10.1016/s0079-6603 (00) 66025-7.
6. Shatkin, A. J. (1976). Capping of eucaryotic mRNAs. Cell 9, 645-653. 10.1016/0092-8674 (76) 90128-8.
7. Decroly, E., Ferron, F., Lescar, J., and Canard, B. (2011). Conventional and unconventional mechanisms for capping viral mRNA. Nat Rev Microbiol 10, 51-65. 10.1038/nrmicro2675.
8. Galloway, A., and Cowling, V. H. (2019). mRNA cap regulation in mammalian cell function and fate. Biochim Biophys Acta Gene Regul Mech 1862, 270-279. 10.1016/j.bbagrm.2018.09.011.
9. Osborne, M. J., and Borden, K. L. (2015). The eukaryotic translation initiation factor eIF4E in the nucleus: taking the road less traveled. Immunol Rev 263, 210-223. 10.1111/imr.12240.
10. Culjkovic, B., Topisirovic, I., and Borden, K. L. (2007). Controlling gene expression through RNA regulons: the role of the eukaryotic translation initiation factor eIF4E. Cell Cycle 6, 65-69. 10.4161/cc.6.1.3688.
11. Trepotec, Z., Geiger, J., Plank, C., Aneja, M. K., and Rudolph, C. (2019). Segmented poly(A) tails significantly reduce recombination of plasmid DNA without affecting mRNA translation efficiency or half-life. RNA 25, 507-518. 10.1261/rna.069286.118.
12. Nicholson, A. L., and Pasquinelli, A. E. (2019). Tales of Detailed Poly (A) Tails. Trends Cell Biol 29, 191-200. 10.1016/j.tcb.2018.11.002.
13. Beverly, M., Hagen, C., and Slack, O. (2018). Poly A tail length analysis of in vitro transcribed mRNA by LC-MS. Anal Bioanal Chem 410, 1667-1677. 10.1007/s00216-017-0840-6.
14. Mangus, D. A., Evans, M. C., and Jacobson, A. (2003). Poly (A)-binding proteins: multifunctional scaffolds for the post-transcriptional control of gene expression. Genome Biol 4, 223. 10.1186/gb-2003-4-7-223.
15. Goldstrohm, A. C., and Wickens, M. (2008). Multifunctional deadenylase complexes diversify mRNA control. Nat Rev Mol Cell Biol 9, 337-344. 10.1038/nrm2370.
16. Nelson, J., Sorensen, E. W., Mintri, S., Rabideau, A. E., Zheng, W., Besin, G., Khatwani, N., Su, S. V., Miracco, E. J., Issa, W. J., Hoge, S., et al. (2020). Impact of mRNA chemistry and manufacturing process on innate immune activation. Sci Adv 6, eaaz6893. 10.1126/sciadv.aaz6893.
17. Rosa, S. S., Prazeres, D. M. F., Azevedo, A. M., and Marques, M. P. C. (2021). mRNA vaccines manufacturing: Challenges and bottlenecks. Vaccine 39, 2190-2200. 10.1016/j.vaccine.2021.03.038.
18. Copur, M. (2021). Messenger RNA Vaccines: Beckoning of a New Era in Cancer Immunotherapy. Oncology (Williston Park) 35, 190-198. 10.46883/ONC.2021.3504.0198.
19. Van Hoecke, L., and Roose, K. (2019). How mRNA therapeutics are entering the monoclonal antibody field. J Transl Med 17, 54. 10.1186/s12967-019-1804-8.
20. Contreras, R., Cheroutre, H., Degrave, W., and Fiers, W. (1982). Simple, efficient in vitro synthesis of capped RNA useful for direct expression of cloned eukaryotic genes. Nucleic Acids Res 10, 6353-6362. 10.1093/nar/10.20.6353.
21. Konarska, M. M., Padgett, R. A., and Sharp, P. A. (1984). Recognition of cap structure in splicing in vitro of mRNA precursors. Cell 38, 731-736. 10.1016/0092-8674 (84) 90268-x.
22. Pelletier, J., and Sonenberg, N. (1985). Insertion mutagenesis to increase secondary structure within the 5′ noncoding region of a eukaryotic mRNA reduces translational efficiency. Cell 40, 515-526. 10.1016/0092-8674 (85) 90200-4.
23. Nielsen, D. A., and Shapiro, D. J. (1986). Preparation of capped RNA transcripts using T7 RNA polymerase. Nucleic Acids Res 14, 5936. 10.1093/nar/14.14.5936.
24. Pasquinelli, A. E., Dahlberg, J. E., and Lund, E. (1995). Reverse 5′ caps in RNAs made in vitro by phage RNA polymerases. RNA 1, 957-967.
25. Paterson, B. M., and Rosenberg, M. (1979). Efficient translation of prokaryotic mRNAs in a eukaryotic cell-free system requires addition of a cap structure. Nature 279, 692-696. 10.1038/279692a0.
26. Green, M. R., Maniatis, T., and Melton, D. A. (1983). Human beta-globin pre-mRNA synthesized in vitro is accurately spliced in Xenopus oocyte nuclei. Cell 32, 681-694. 10.1016/0092-8674 (83) 90054-5.
27. Fuchs, A. L., Neu, A., and Sprangers, R. (2016). A general method for rapid and cost-efficient large-scale production of 5′ capped RNA. RNA 22, 1454-1466. 10.1261/rna.056614.116.
28. Ray, D., Shah, A., Tilgner, M., Guo, Y., Zhao, Y., Dong, H., Deas, T. S., Zhou, Y., Li, H., and Shi, P. Y. (2006). West Nile virus 5′-cap structure is formed by sequential guanine N-7 and ribose 2′-O methylations by nonstructural protein 5. J Virol 80, 8362-8370. 10.1128/JVI.00814-06.
29. Preiss, T., Muckenthaler, M., and Hentze, M. W. (1998). Poly (A)-tail-promoted translation in yeast: implications for translational control. RNA 4, 1321-1331. 10.1017/s1355838298980669.
30. Grier, A. E., Burleigh, S., Sahni, J., Clough, C. A., Cardot, V., Choe, D. C., Krutein, M. C., Rawlings, D. J., Jensen, M. C., Scharenberg, A. M., and Jacoby, K. (2016). pEVL: A Linear Plasmid for Generating mRNA IVT Templates With Extended Encoded Poly (A) Sequences. Mol Ther Nucleic Acids 5, e306. 10.1038/mtna.2016.21.
31. Cao, G. J., and Sarkar, N. (1992). Identification of the gene for an Escherichia coli poly(A) polymerase. Proc Natl Acad Sci USA 89, 10380-10384. 10.1073/pnas.89.21.10380.
32. Martin, G., and Keller, W. (1998). Tailing and 3′-end labeling of RNA with yeast poly(A) polymerase and various nucleotides. RNA 4, 226-230.
33. Weissman, D. (2015). mRNA transcript therapy. Expert Rev Vaccines 14, 265-281. 10.1586/14760584.2015.973859.
34. Jang, S. K., Krausslich, H. G., Nicklin, M. J., Duke, G. M., Palmenberg, A. C., and Wimmer, E. (1988). A segment of the 5′ nontranslated region of encephalomyocarditis virus RNA directs internal entry of ribosomes during in vitro translation. J Virol 62, 2636-2643. 10.1128/JVI.62.8.2636-2643.1988.
35. Baird, S. D., Turcotte, M., Korneluk, R. G., and Holcik, M. (2006). Searching for IRES. RNA 12, 1755-1785. 10.1261/rna.157806.
36. Renaud-Gabardos, E., Hantelys, F., Morfoisse, F., Chaufour, X., Garmy-Susini, B., and Prats, A. C. (2015). Internal ribosome entry site-based vectors for combined gene therapy. World J Exp Med 5, 11-20. 10.5493/wjem.v5.il.11.
37. van den Akker, G. G. H., Zacchini, F., Housmans, B. A. C., van der Vloet, L., Caron, M. M. J., Montanaro, L., and Welting, T. J. M. (2021). Current Practice in Bicistronic IRES Reporter Use: A Systematic Review. Int J Mol Sci 22. 10.3390/ijms22105193.
38. Pestova, T. V., Hellen, C. U., and Shatsky, I. N. (1996). Canonical eukaryotic initiation factors determine initiation of translation by internal ribosomal entry. Mol Cell Biol 16, 6859-6869. 10.1128/MCB.16.12.6859.
39. Emory, S. A., Bouvet, P., and Belasco, J. G. (1992). A 5′-terminal stem-loop structure can stabilize mRNA in Escherichia coli. Genes Dev 6, 135-148. 10.1101/gad.6.1.135.
40. Deutscher, M. P. (2006). Degradation of RNA in bacteria: comparison of mRNA and stable RNA. Nucleic Acids Res 34, 659-666. 10.1093/nar/gkj472.
41. Houseley, J., and Tollervey, D. (2009). The many pathways of RNA degradation. Cell 136, 763-776. 10.1016/j.cell.2009.01.019.
42. Shirokikh, N. E., and Spirin, A. S. (2008). Poly (A) leader of eukaryotic mRNA bypasses the dependence of translation on initiation factors. Proc Natl Acad Sci USA 105, 10738-10743. 10.1073/pnas.0804940105.
43. Wigington, C. P., Williams, K. R., Meers, M. P., Bassell, G. J., and Corbett, A. H. (2014). Poly (A) RNA-binding proteins and polyadenosine RNA: new members and novel functions. Wiley Interdiscip Rev RNA 5, 601-622. 10.1002/wrna.1233.
44. Tusup, M., French, L. E., De Matos, M., Gatfield, D., Kundig, T., and Pascolo, S. (2019). Design of in vitro Transcribed mRNA Vectors for Research and Therapy. Chimia (Aarau) 73, 391-394. 10.2533/chimia.2019.391.
45. Akiyama, B. M., Eiler, D., and Kieft, J. S. (2016). Structured RNAs that evade or confound exonucleases: function follows form. Curr Opin Struct Biol 36, 40-47. 10.1016/j.sbi.2015.12.006.
46. Maruggi, G., Zhang, C., Li, J., Ulmer, J. B., and Yu, D. (2019). mRNA as a Transformative Technology for Vaccine Development to Control Infectious Diseases. Mol Ther 27, 757-772. 10.1016/j.ymthe.2019.01.020.
47. Spriggs, K. A., Bushell, M., Mitchell, S. A., and Willis, A. E. (2005). Internal ribosome entry segment-mediated translation during apoptosis: the role of IRES-trans-acting factors. Cell Death Differ 12, 585-591. 10.1038/sj.cdd.4401642.
48. Komar, A. A., and Hatzoglou, M. (2011). Cellular IRES-mediated translation: the war of ITAFs in pathophysiological states. Cell Cycle 10, 229-240. 10.4161/cc.10.2.14472.
49. Lee, K. M., Chen, C. J., and Shih, S. R. (2017). Regulation Mechanisms of Viral IRES-Driven Translation. Trends Microbiol 25, 546-561. 10.1016/j.tim.2017.01.010.
50. Bradrick, S. S., Dobrikova, E. Y., Kaiser, C., Shveygert, M., and Gromeier, M. (2007). Poly (A)-binding protein is differentially required for translation mediated by viral internal ribosome entry sites. RNA 13, 1582-1593. 10.1261/rna.556107.
51. Wesselhoeft, R. A., Kowalski, P. S., and Anderson, D. G. (2018). Engineering circular RNA for potent and stable translation in eukaryotic cells. Nat Commun 9, 2629. 10.1038/s41467-018-05096-6.
52. Chen, Y. G., Kim, M. V., Chen, X., Batista, P. J., Aoyama, S., Wilusz, J. E., Iwasaki, A., and Chang, H. Y. (2017). Sensing Self and Foreign Circular RNAs by Intron Identity. Mol Cell 67, 228-238 e225. 10.1016/j.molcel.2017.05.022.
53. Svoboda, P., and Di Cara, A. (2006). Hairpin RNA: a secondary structure of primary importance. Cell Mol Life Sci 63, 901-908. 10.1007/s00018-005-5558-5.
54. Unniraman, S., Chatterji, M., and Nagaraja, V. (2002). A hairpin near the 5′ end stabilises the DNA gyrase mRNA in Mycobacterium smegmatis. Nucleic Acids Res 30, 5376-5381. 10.1093/nar/gkf697.
55. Regnier, P., and Arraiano, C. M. (2000). Degradation of mRNA in bacteria: emergence of ubiquitous features. Bioessays 22, 235-244. 10.1002/(SICI) 1521-1878 (200003) 22: 3<235::AID-BIES5>3.0.CO; 2-2.
56. Belasco, J. G., Nilsson, G., von Gabain, A., and Cohen, S. N. (1986). The stability of E. coli gene transcripts is dependent on determinants localized to specific mRNA segments. Cell 46, 245-251. 10.1016/0092-8674 (86) 90741-5.
57. Hansen, M. J., Chen, L. H., Fejzo, M. L., and Belasco, J. G. (1994). The ompA 5′ untranslated region impedes a major pathway for mRNA degradation in Escherichia coli. Mol Microbiol 12, 707-716. 10.1111/j.1365-2958.1994.tb01058.x.
58. Agaisse, H., and Lereclus, D. (1996). STAB-SD: a Shine-Dalgarno sequence in the 5′ untranslated region is a determinant of mRNA stability. Mol Microbiol 20, 633-643. 10.1046/j.1365-2958.1996.5401046.x.
59. Marzluff, W. F., Wagner, E. J., and Duronio, R. J. (2008). Metabolism and regulation of canonical histone mRNAs: life without a poly(A) tail. Nat Rev Genet 9, 843-854. 10.1038/nrg2438.
60. Martinez-Salas, E., Francisco-Velilla, R., Fernandez-Chamorro, J., and Embarek, A. M. (2017). Insights into Structural and Mechanistic Features of Viral IRES Elements. Front Microbiol 8, 2629. 10.3389/fmicb.2017.02629.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, materials, and arrangements of the parts and method stages which have been described and illustrated in order to explain the nature of this inventive subject matter can be made without departing from the principles and scope of the inventive subject matter.

A biologically functional polynucleotide consisting of,
a mRNA transcript having a first end and a second end,
   wherein a first terminal hairpin is located at said first end of said mRNA transcript,
   wherein a second terminal hairpin is located at said second end of said mRNA transcript,
   wherein said first terminal hairpin and said second terminal hairpin is a nucleotide sequence configured to stably fold onto itself via nucleotide pairing, and
   wherein said first terminal hairpin and said second terminal hairpin are linked to said first end and said second end via no more than two unpaired nucleotides.

A biologically functional polynucleotide consisting of,
a chemically synthesized mRNA molecule having a first end and a second end,
   wherein a first terminal hairpin is located at said first end of said chemically synthesized mRNA molecule,
   wherein a second terminal hairpin is located at said second end of said chemically synthesized mRNA molecule,
   wherein said first terminal hairpin and said second terminal hairpin are nucleotide sequences configured to stably fold onto themselves via nucleotide pairing, and
   wherein said first terminal hairpin and said second terminal hairpin are linked to said first end and said second end via no more than two unpaired nucleotides.

A biologically functional polynucleotide consisting of,
a mRNA transcript having a 5′ end and a 3′ end,
   wherein a first terminal hairpin is located at said 3′ end of said mRNA transcript,
   wherein said first terminal hairpin is a nucleotide sequence configured to stably fold onto itself via nucleotide pairing,
   wherein said first terminal hairpin is linked to said 3′ end via no more than two unpaired nucleotides, and
a cap operably linked to said 5′ end of said mRNA transcript.

A biologically functional polynucleotide consisting of,
a chemically synthesized mRNA molecule having a 5′ end and a 3′ end,
   wherein a first terminal hairpin is located at said 3′ end of said chemically synthesized mRNA molecule,
   wherein said first terminal hairpin is a nucleotide sequence configured to stably fold onto itself via nucleotide pairing,
   wherein said first terminal hairpin is linked to said 3′ end via no more than two unpaired nucleotides, and
a cap operably linked to said 5′ end of said chemically synthesized mRNA molecule.

A biologically functional polynucleotide consisting of,
a mRNA transcript having a 5′ end and a 3′ end,
   wherein a first terminal hairpin is located at said 5′ end of said mRNA transcript,
   wherein a poly(A) tail sequence is located at said 3′ end of said mRNA transcript,
   wherein said first terminal hairpin is a nucleotide sequence configured to stably fold onto itself via nucleotide pairing, and
   wherein said first terminal hairpin is linked to said 5′ end via no more than two unpaired nucleotides.

A biologically functional polynucleotide consisting of,
a chemically synthesized mRNA molecule having a 5′ end and a 3′ end,
   wherein a first terminal hairpin is located at said 5′ end of said chemically synthesized mRNA molecule,
   wherein a poly(A) tail sequence is located at said 3′ end of said chemically synthesized mRNA molecule,
   wherein said first terminal hairpin is a nucleotide sequence configured to stably fold onto itself via nucleotide pairing, and
   wherein said first terminal hairpin is linked to said 5′ end via no more than two unpaired nucleotides.

A DNA template configured to encode a target RNA transcript consisting of,
a DNA source configured to create a target mRNA transcript,
   wherein said target mRNA transcript comprises a first end and a second end,
   wherein a first terminal hairpin is located at said first end of said mRNA transcript,
   wherein a second terminal hairpin is located at said second end of said mRNA transcript,
   wherein said first terminal hairpin and said second terminal hairpin are nucleotide sequences configured to stably fold onto themselves via nucleotide pairing, and
   wherein said first terminal hairpin and said second terminal hairpin are linked to said first end and said second end via no more than two unpaired nucleotides.

A DNA template configured to encode a target RNA transcript consisting of,
a DNA source configured to create a target mRNA transcript,
   wherein said target mRNA transcript comprises a first end and a second end,
   wherein a first terminal hairpin is located at said first end of said mRNA transcript,
   wherein a poly(A) tail is located at said second end of said mRNA transcript,
   wherein said first terminal hairpin is a nucleotide sequence configured to stably fold onto themselves via nucleotide pairing, and
   wherein said first terminal hairpin is linked to said first end via no more than two unpaired nucleotides.

A DNA template configured to encode a target RNA transcript consisting of,
a DNA source configured to create a target mRNA transcript,
   wherein said target mRNA transcript comprises a 5′ end and a 3′ end,
   wherein a first terminal hairpin is located at said 3′ end of said mRNA transcript,
   wherein a cap is located at said 5′ end of said mRNA transcript,
   wherein said first terminal hairpin is a nucleotide sequence configured to stably fold onto themselves via nucleotide pairing, and
   wherein said first terminal hairpin is linked to said first end via no more than two unpaired nucleotides.

A method for creating a biologically functional polynucleotide comprising steps of:
generating a target mRNA vector having an mRNA transcript possessing a first end and a second end,
   wherein a first terminal hairpin is located at said first end of said mRNA transcript,
   wherein a second terminal hairpin is located at said second end of said mRNA transcript,
   wherein said first terminal hairpin and said second terminal hairpin are nucleotide sequences configured to stably fold onto themselves via nucleotide pairing, and
   wherein said first terminal hairpin and said second terminal hairpin are linked to said first end and said second end via no more than two unpaired nucleotides,
   wherein said target mRNA vector has one of a mRNA transcript and a chemically synthesized mRNA molecule,
inserting said target mRNA vector into a DNA source to create a DNA template,
performing in vitro transcription on said DNA template using DNA-dependent RNA polymerase to obtain a target mRNA vector product, and
purifying said target mRNA vector product to produce therapeutic exogenous mRNA.

A method for creating a biologically functional polynucleotide comprising steps of:
generating a target mRNA vector having a chemically synthesized mRNA molecule possessing a first end and a second end,
   wherein a first terminal hairpin is located at said first end of said chemically synthesized mRNA molecule,
   wherein a second terminal hairpin is located at said second end of said chemically synthesized mRNA molecule,
   wherein said first terminal hairpin and said second terminal hairpin are nucleotide sequences configured to stably fold onto themselves via nucleotide pairing, and
   wherein said first terminal hairpin and said second terminal hairpin are linked to said first end and said second end via no more than two unpaired nucleotides,
purifying said target mRNA vector product to produce therapeutic exogenous mRNA.

A method for creating a biologically functional polynucleotide comprising steps of:
generating a target mRNA vector having an mRNA transcript possessing a 5′ end and a 3′ end,
   wherein a first terminal hairpin is located at said 3′ end of said mRNA transcript,
   wherein a cap is located at said 5′ end of said mRNA transcript,
   wherein said first terminal hairpin is a nucleotide sequence configured to stably fold onto itself via nucleotide pairing,
   wherein said first terminal hairpin is linked to said 3′ end via no more than two unpaired nucleotides,
inserting said target mRNA vector into a DNA source to create a DNA template,
performing in vitro transcription on said DNA template using DNA-dependent RNA polymerase to obtain a target mRNA vector product, and
purifying said target mRNA vector product to produce therapeutic exogenous mRNA.

A method for creating a biologically functional polynucleotide comprising steps of:
generating a target mRNA vector having an mRNA transcript possessing a 5′ end and a 3′ end,
   wherein a first terminal hairpin is located at said 5′ end of said mRNA transcript,
   wherein a poly(A) tail is located at said 3′ end of said mRNA transcript,
   wherein said first terminal hairpin is a nucleotide sequence configured to stably fold onto itself via nucleotide pairing,
   wherein said first terminal hairpin is linked to said 3′ end via no more than two unpaired nucleotides,
inserting said target mRNA vector into a DNA source to create a DNA template,
performing in vitro transcription on said DNA template using DNA-dependent RNA polymerase to obtain a target mRNA vector product, and
purifying said target mRNA vector product to produce therapeutic exogenous mRNA.

A method for creating a biologically functional polynucleotide comprising steps of:
generating a target mRNA vector having a chemically synthesized mRNA molecule possessing a 5′ end and a 3′ end,
   wherein a first terminal hairpin is located at said 3′ end of said mRNA transcript,
   wherein a cap is located at said 5′ end of said mRNA transcript,
   wherein said first terminal hairpin is a nucleotide sequence configured to stably fold onto itself via nucleotide pairing,
   wherein said first terminal hairpin is linked to said 3′ end via no more than two unpaired nucleotides,
obtaining a target mRNA vector product using said target mRNA vector, and
purifying said target mRNA vector product to produce therapeutic exogenous mRNA.

A method for creating a biologically functional polynucleotide comprising steps of:
generating a target mRNA vector having a chemically synthesized mRNA molecule possessing a 5′ end and a 3′ end,
   wherein a first terminal hairpin is located at said 5′ end of said mRNA transcript,
   wherein a poly(A) tail is located at said 3′ end of said mRNA transcript,
   wherein said first terminal hairpin is a nucleotide sequence configured to stably fold onto itself via nucleotide pairing,
   wherein said first terminal hairpin is linked to said 3′ end via no more than two unpaired nucleotides,
obtaining a target mRNA vector product using said target mRNA vector, and
purifying said target mRNA vector product to produce therapeutic exogenous mRNA.

A product for use as a therapeutic agent, wherein said product is prepared by:
inserting a target mRNA vector sequence into a DNA source to create a DNA template,
   wherein said target mRNA vector sequence encodes a first terminal hairpin that is located at a first end of said target mRNA vector and a second terminal hairpin that is located at a second end of said target mRNA vector,
   wherein an internal translation initiation site is incorporated into said target mRNA vector,
performing in vitro transcription on said DNA template using DNA-dependent RNA polymerase to obtain a target mRNA vector product, and
purifying said target mRNA vector product to produce a therapeutic agent,
   wherein said therapeutic agent is said product.

A product for use as a therapeutic agent, wherein said product is prepared by:
inserting a target mRNA vector sequence into a DNA source to create a DNA template,
   wherein said target mRNA vector sequence encodes a first terminal hairpin onto a 5′ end of said target mRNA vector and a poly(A) tail at a 3′ end of said target mRNA vector,
   wherein an internal translation initiation site is incorporated into said target mRNA vector,
purifying said target mRNA vector product to produce a therapeutic agent,
   wherein said therapeutic agent is said product.

A product for use as a therapeutic agent, wherein said product is prepared by:
inserting a target mRNA vector sequence into a DNA source to create a DNA template,
   wherein said target mRNA vector sequence encodes a first terminal hairpin onto a 3′ end of said target mRNA vector and a cap at a 5′ end of said target mRNA vector,
   wherein an internal translation initiation site is incorporated into said target mRNA vector,
performing in vitro transcription on said DNA template using DNA-dependent RNA polymerase to obtain a target mRNA vector product, and
performing in vitro transcription on said DNA template using DNA-dependent RNA polymerase to obtain a target mRNA vector product, and
purifying said target mRNA vector product to produce a therapeutic agent,
   wherein said therapeutic agent is said product.

A product for use as a therapeutic agent, wherein said product is prepared by:
performing chemical synthesis on an mRNA vector having a target mRNA vector sequence to create a chemically synthesized mRNA molecule,
   wherein said mRNA vector has a first end and a second end,
   wherein said target mRNA vector sequence encodes a first terminal hairpin that is located at a first end of said target mRNA vector and a second terminal hairpin that is located at a second end of said target mRNA vector,
   wherein an internal translation initiation site is incorporated into said chemically synthesized mRNA molecule,
performing transcription on said chemically synthesized mRNA molecule to obtain a target mRNA vector product,
purifying said target mRNA vector product to produce a therapeutic agent,
   wherein said therapeutic agent is said product.

A product for use as a therapeutic agent, wherein said product is prepared by:
performing chemical synthesis on an mRNA vector having a target mRNA vector sequence to create a chemically synthesized mRNA molecule,
   wherein said target mRNA vector sequence encodes a first terminal hairpin at a 5′ end of said target mRNA vector and a poly(A) tail at a 3′ end of said target mRNA vector,
   wherein an internal translation initiation site is incorporated into said chemically synthesized mRNA molecule,
performing transcription on said chemically synthesized mRNA molecule to obtain a target mRNA vector product,
purifying said target mRNA vector product to produce a therapeutic agent,
   wherein said therapeutic agent is said product.

A product for use as a therapeutic agent, wherein said product is prepared by:
performing chemical synthesis on an mRNA vector having a target mRNA vector sequence to create a chemically synthesized mRNA molecule,
   wherein said target mRNA vector sequence encodes a first terminal hairpin at a 3′ end of said target mRNA vector and a cap at a 5′ end of said target mRNA vector,
   wherein an internal translation initiation site is incorporated into said chemically synthesized mRNA molecule,
performing transcription on said chemically synthesized mRNA molecule to obtain a target mRNA vector product,
purifying said target mRNA vector product to produce a therapeutic agent,
wherein said therapeutic agent is said product.

What is claimed is:

1. A biologically functional polynucleotide comprising,
a mRNA transcript having a first end and a second end and lacking a cap and a poly(A) tail,
wherein a first terminal hairpin structure is located at said first end of said mRNA transcript,
wherein a second terminal hairpin structure is located at said second end of said mRNA transcript,
wherein said first terminal hairpin structure and said second terminal hairpin structure is a nucleotide sequence configured to stably fold onto itself via nucleotide pairing, and
wherein said first terminal hairpin structure and said second terminal hairpin structure are linked to said first end and said second end via no more than two unpaired nucleotides,
wherein said first terminal hairpin structure has a number of individual hairpins that is at least equal to a number of individual hairpins of said second terminal hairpin structure,
wherein said number of individual hairpins of said first terminal hairpin structure determines an amount of product when said mRNA transcript undergoes translation.

2. The biologically functional polynucleotide of claim 1, further comprising a cap at a 5′ end, wherein said 5′ end is one of said first end or said second end, wherein one of said first terminal hairpin structure or said second terminal hairpin structure is secured downstream to said cap.

3. The biologically functional polynucleotide of claim 1, further comprising a poly(A) tail at a 3′ end, wherein said 3′ end is one of said first end or said second end, wherein one of said first terminal hairpin structure or said second terminal hairpin structure is secured upstream to said poly(A) tail.

4. The biologically functional polynucleotide of claim 1, further comprising a cap at said first end and a poly(A) tail at said second end, wherein said first end is a 5′ end and said second end is a 3′ end, wherein said first terminal hairpin structure is secured downstream of said cap and said second terminal hairpin structure is secured upstream of said poly(A) tail.

5. The biologically functional polynucleotide of claim 1, wherein said first terminal hairpin structure and said second terminal hairpin structure are one of a triple hairpin structure, double hairpin structure, and single hairpin structure,
wherein said double hairpin structure and said triple hairpin structure comprise no more than two unpaired nucleotides between said individual hairpins.

6. The biologically functional polynucleotide of claim 1, further comprising an internal translation initiation site that is incorporated into said mRNA transcript.

7. The biologically functional polynucleotide of claim 6, wherein said internal translation initiation site is an internal ribosome entry site (IRES).

8. The biologically functional polynucleotide of claim 7, further comprising a delivered gene sequence that is incorporated into said mRNA transcript.

9. The biologically functional polynucleotide of claim 8, wherein said internal translation initiation site is incorporated into said mRNA transcript at a location that is upstream of said delivered gene sequence of said mRNA transcript.

10. The biologically functional polynucleotide of claim 9, wherein an internal translation initiation site is incorporated into said mRNA transcript at a 5′ untranslated region (5′ UTR) of said mRNA transcript.

11. The biologically functional polynucleotide of claim 1, further comprising a DNA source,
wherein a nucleotide sequence containing instructions for said mRNA transcript is inserted within said DNA source to create a DNA template,
wherein said DNA template is configured to encode RNA transcripts containing said mRNA transcript.

12. The biologically functional polynucleotide of claim 11, wherein said DNA source is one of a plasmid DNA, genomic DNA, synthetic DNA, and PCR product.

13. A biologically functional polynucleotide comprising,
a chemically synthesized mRNA molecule having a first end and a second end and lacking a cap and a poly(A) tail,
wherein a first terminal hairpin structure is located at said first end of said chemically synthesized mRNA molecule,
wherein a second terminal hairpin structure is located at said second end of said chemically synthesized mRNA molecule,
wherein said first terminal hairpin structure and said second terminal hairpin structure are nucleotide sequences configured to stably fold onto themselves via nucleotide pairing, and
wherein said first terminal hairpin structure and said second terminal hairpin structure are linked to said first end and said second end via no more than two unpaired nucleotides,
wherein said first terminal hairpin structure has a number of individual hairpins that is at least equal to a number of individual hairpins of said second terminal hairpin structure,
wherein said number of individual hairpins of said first terminal hairpin structure determines an amount of product when said chemically synthesized mRNA molecule undergoes translation.

14. The biologically functional polynucleotide of claim 13, further comprising a cap at a 5′ end, wherein said 5′ end is one of said first end or said second end, wherein one of said first terminal hairpin structure or said second terminal hairpin structure is secured downstream to said cap.

15. The biologically functional polynucleotide of claim 13, further comprising a poly(A) tail at a 3′ end, wherein said 3′ end is one of said first end or said second end, wherein one of said first terminal hairpin structure or said second terminal hairpin structure is secured upstream to said poly(A) tail.

16. The biologically functional polynucleotide of claim 13, further comprising a cap at said first end and a poly(A) tail at said second end, wherein said first end is a 5′ end and said second end is a 3′ end, wherein said first terminal hairpin structure is secured downstream of said cap and said second terminal hairpin structure is secured upstream of said poly(A) tail.

17. The biologically functional polynucleotide of claim 13, wherein said first terminal hairpin structure and said second terminal hairpin structure are one of a triple hairpin structure, double hairpin structure, and single hairpin structure,
wherein said double hairpin structure and said triple hairpin structure comprise no more than two unpaired nucleotides between said individual hairpins.

18. The biologically functional polynucleotide of claim 13, further comprising an internal translation initiation site that is incorporated into said chemically synthesized mRNA molecule.

19. The biologically functional polynucleotide of claim 18, wherein said internal translation initiation site is an internal ribosome entry site (IRES).

20. The biologically functional polynucleotide of claim 19, further comprising a delivered gene sequence that is incorporated into said chemically synthesized mRNA molecule.

21. The biologically functional polynucleotide of claim 20, wherein said internal translation initiation site is incorporated into said chemically synthesized mRNA molecule at a location that is upstream of said delivered gene sequence of said chemically synthesized mRNA molecule.

22. The biologically functional polynucleotide of claim 21, wherein an internal translation initiation site is incorporated into said chemically synthesized mRNA molecule at a 5′ untranslated region (5′ UTR) of said chemically synthesized mRNA molecule.

23. A biologically functional polynucleotide comprising,
a mRNA transcript having a 5′ end and a 3′ end and lacks a poly(A) tail,
wherein a first terminal hairpin structure is located at said 3′ end of said mRNA transcript,
wherein said first terminal hairpin structure is a nucleotide sequence configured to stably fold onto itself via nucleotide pairing,
wherein a number of individual hairpins of said first terminal hairpin structure determines an amount of product when said mRNA transcript undergoes translation,
wherein said first terminal hairpin structure is linked to said 3′ end via no more than two unpaired nucleotides, and
a cap operably linked to said 5′ end of said mRNA transcript.

24. The biologically functional polynucleotide of claim 23, further comprising a poly(A) tail at the said 3′ end, wherein said first terminal hairpin structure is secured upstream to said poly(A) tail.

25. The biologically functional polynucleotide of claim 23, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

26. The biologically functional polynucleotide of claim 23, further comprising an internal translation initiation site that is incorporated into said mRNA transcript.

27. The biologically functional polynucleotide of claim 26, wherein said internal translation initiation site is an internal ribosome entry site (IRES).

28. The biologically functional polynucleotide of claim 27, further comprising a delivered gene sequence that is incorporated into said mRNA transcript.

29. The biologically functional polynucleotide of claim 28, wherein said internal translation initiation site is incorporated into said mRNA transcript at a location that is upstream of said delivered gene sequence of said mRNA transcript.

30. The biologically functional polynucleotide of claim 29, wherein an internal translation initiation site is incorporated into said mRNA transcript at a 5′ untranslated region (5′ UTR) of said mRNA transcript.

31. The biologically functional polynucleotide of claim 23, further comprising a DNA source,
wherein a nucleotide sequence containing instructions for said mRNA transcript is inserted within said DNA source to create a DNA template,
wherein said DNA template is configured to encode RNA transcripts containing said mRNA transcript.

32. The biologically functional polynucleotide of claim 31, wherein said DNA source is one of a plasmid DNA, genomic DNA, synthetic DNA, and PCR product.

33. A biologically functional polynucleotide comprising,
a chemically synthesized mRNA molecule having a 5′ end and a 3′ end and lacks a poly(A) tail,
wherein a first terminal hairpin structure is located at said 3′ end of said chemically synthesized mRNA molecule,
wherein said first terminal hairpin structure is a nucleotide sequence configured to stably fold onto itself via nucleotide pairing,
wherein a number of individual hairpins of said first terminal hairpin structure determines an amount of product when said chemically synthesized mRNA molecule undergoes translation,
wherein said first terminal hairpin structure is linked to said 3′ end via no more than two unpaired nucleotides, and
a cap operably linked to said 5′ end of said chemically synthesized mRNA molecule.

34. The biologically functional polynucleotide of claim 33, further comprising a poly(A) tail at the said 3′ end, wherein said first terminal hairpin structure is secured upstream to said poly(A) tail.

35. The biologically functional polynucleotide of claim 33, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

36. The biologically functional polynucleotide of claim 33, further comprising an internal translation initiation site that is incorporated into said chemically synthesized mRNA molecule.

37. The biologically functional polynucleotide of claim 36, wherein said internal translation initiation site is an internal ribosome entry site (IRES).

38. The biologically functional polynucleotide of claim 37, further comprising a delivered gene sequence that is incorporated into said chemically synthesized mRNA molecule.

39. The biologically functional polynucleotide of claim 38, wherein said internal translation initiation site is incorporated into said chemically synthesized mRNA molecule at a location that is upstream of said delivered gene sequence of said chemically synthesized mRNA molecule.

40. The biologically functional polynucleotide of claim 39, wherein an internal translation initiation site is incorporated into said chemically synthesized mRNA molecule at a 5′ untranslated region (5′ UTR) of said chemically synthesized mRNA molecule.

41. A biologically functional polynucleotide comprising,
a mRNA transcript having a 5′ end and a 3′ end and lacking a cap,
wherein a first terminal hairpin structure is located at said 5′ end of said mRNA transcript,
wherein a poly(A) tail sequence is located at said 3′ end of said mRNA transcript,
wherein a number of individual hairpins of said first terminal hairpin structure determines an amount of product when said mRNA transcript undergoes translation,
wherein said first terminal hairpin structure is a nucleotide sequence configured to stably fold onto itself via nucleotide pairing, and
wherein said first terminal hairpin structure is linked to said 5′ end via no more than two unpaired nucleotides.

42. The biologically functional polynucleotide of claim 41, wherein a poly(A) tail is added to a 3′ end of said mRNA transcript using a poly(A) polymerase.

43. The biologically functional polynucleotide of claim 41, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

44. The biologically functional polynucleotide of claim 41, further comprising an internal translation initiation site that is incorporated into said mRNA transcript.

45. The biologically functional polynucleotide of claim 44, wherein said internal translation initiation site is an internal ribosome entry site (IRES).

46. The biologically functional polynucleotide of claim 45 further comprising a delivered gene sequence that is incorporated into said mRNA transcript.

47. The biologically functional polynucleotide of claim 46, wherein said internal translation initiation site is incorporated into said mRNA transcript at a location that is upstream of said delivered gene sequence of said mRNA transcript.

48. The biologically functional polynucleotide of claim 47, wherein an internal translation initiation site is incorporated into said mRNA transcript at a 5′ untranslated region (5′ UTR) of said mRNA transcript.

49. The biologically functional polynucleotide of claim 41, further comprising a DNA source,
wherein a nucleotide sequence containing instructions for said mRNA transcript is inserted within said DNA source to create a DNA template,
wherein said DNA template is configured to encode RNA transcripts containing said mRNA transcript.

50. The biologically functional polynucleotide of claim 49, wherein said DNA source is one of a plasmid DNA, genomic DNA, synthetic DNA, and PCR product.

51. A biologically functional polynucleotide comprising,
a chemically synthesized mRNA molecule having a 5′ end and a 3′ end and lacking a cap,
wherein a first terminal hairpin structure is located at said 5′ end of said chemically synthesized mRNA molecule,
wherein a poly(A) tail sequence is located at said 3′ end of said chemically synthesized mRNA molecule,
wherein a number of individual hairpins of said first terminal hairpin structure determines an amount of product when said chemically synthesized mRNA molecule undergoes translation,
wherein said first terminal hairpin structure is a nucleotide sequence configured to stably fold onto itself via nucleotide pairing, and
wherein said first terminal hairpin structure is linked to said 5′ end via no more than two unpaired nucleotides.

52. The biologically functional polynucleotide of claim 51, wherein a poly(A) tail is added to a 3′ end of said chemically synthesized mRNA molecule using a poly(A) polymerase.

53. The biologically functional polynucleotide of claim 51, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

54. The biologically functional polynucleotide of claim 51, further comprising an internal translation initiation site that is incorporated into said chemically synthesized mRNA molecule.

55. The biologically functional polynucleotide of claim 54, wherein said internal translation initiation site is an internal ribosome entry site (IRES).

56. The biologically functional polynucleotide of claim 55, further comprising a delivered gene sequence that is incorporated into said chemically synthesized mRNA molecule.

57. The biologically functional polynucleotide of claim 56, wherein said internal translation initiation site is incorporated into said chemically synthesized mRNA molecule at a location that is upstream of said delivered gene sequence of said chemically synthesized mRNA molecule.

58. The biologically functional polynucleotide of claim 57, wherein an internal translation initiation site is incorporated into said chemically synthesized mRNA molecule at a 5′ untranslated region (5′ UTR) of said chemically synthesized mRNA molecule.

59. A DNA template configured to encode a target RNA transcript comprising,
a DNA source configured to create a target mRNA transcript,
wherein said target mRNA transcript comprises a first end and a second end and lacks a cap and a poly(A) tail,
wherein a first terminal hairpin structure is located at said first end of said target mRNA transcript,
wherein a second terminal hairpin structure is located at said second end of said target mRNA transcript,
wherein said first terminal hairpin structure and said second terminal hairpin structure are nucleotide sequences configured to stably fold onto themselves via nucleotide pairing, and
wherein said first terminal hairpin structure and said second terminal hairpin structure are linked to said first end and said second end via no more than two unpaired nucleotides,
wherein said first terminal hairpin structure has a number of individual hairpins that is at least equal to a number of individual hairpins of said second terminal hairpin structure,
wherein said number of individual hairpins of said first terminal hairpin structure determines an amount of product when said mRNA transcript undergoes translation.

60. The DNA template of claim 59, wherein said first terminal hairpin structure and said second terminal hairpin structure are one of a triple hairpin structure, double hairpin structure, and single hairpin structure,
wherein said double hairpin structure and said triple hairpin structure comprise no more than two unpaired nucleotides between said individual hairpins.

61. The DNA template of claim 59, wherein an internal translation initiation site is incorporated into said target mRNA transcript.

62. The DNA template of claim 61, wherein said internal translation initiation site is an internal ribosome entry site (IRES).

63. The DNA template of claim 62, further comprising a delivered gene sequence incorporated into said target mRNA transcript.

64. The DNA template of claim 63, wherein said internal translation initiation site is incorporated into said target mRNA transcript upstream of said delivered gene sequence of said target mRNA transcript.

65. The DNA template of claim 64, wherein said internal translation initiation site is incorporated into said target mRNA transcript at a 5′ untranslated region (5′ UTR) of said target mRNA transcript.

66. The DNA template of claim 59, wherein said DNA source is one of a plasmid DNA, genomic DNA, synthetic DNA, and PCR product.

67. A DNA template configured to encode a target RNA transcript comprising,
a DNA source configured to create a target mRNA transcript,
wherein said target mRNA transcript comprises a first end and a second end and lacks a cap,
wherein a first terminal hairpin structure is located at said first end of said target mRNA transcript,
wherein a poly(A) tail is located at said second end of said target mRNA transcript,
wherein a number of individual hairpins of said first terminal hairpin structure determines an amount of product when said mRNA transcript undergoes translation,
wherein said first terminal hairpin structure is a nucleotide sequence configured to stably fold onto themselves via nucleotide pairing, and
wherein said first terminal hairpin structure is linked to said first end via no more than two unpaired nucleotides.

68. The DNA template of claim 67, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

69. The DNA template of claim 67, wherein an internal translation initiation site is incorporated into said target mRNA transcript.

70. The DNA template of claim 69, wherein said internal translation initiation site is an internal ribosome entry site (IRES).

71. The DNA template of claim 70, further comprising a delivered gene sequence incorporated into said target mRNA transcript.

72. The DNA template of claim 71, wherein said internal translation initiation site is incorporated into said target mRNA transcript upstream of said delivered gene sequence of said target mRNA transcript.

73. The DNA template of claim 72, wherein said internal translation initiation site is incorporated into said target mRNA transcript at a 5′ untranslated region (5′ UTR) of target mRNA transcript.

74. The DNA template of claim 67, wherein said DNA source is one of a plasmid DNA, genomic DNA, synthetic DNA, and PCR product.

75. A DNA template configured to encode a target RNA transcript comprising,
a DNA source configured to create a target mRNA transcript,
wherein said target mRNA transcript comprises a 5′ end and a 3′ end and lacks a poly(A) tail,
wherein a first terminal hairpin structure is located at said 3′ end of said target mRNA transcript,
wherein a cap is located at said 5′ end of said target mRNA transcript,
wherein a number of individual hairpins of said first terminal hairpin structure determines an amount of product when said mRNA transcript undergoes translation,
wherein said first terminal hairpin structure is a nucleotide sequence configured to stably fold onto themselves via nucleotide pairing, and
wherein said first terminal hairpin structure is linked to said 3′ end via no more than two unpaired nucleotides.

76. A method for creating a biologically functional polynucleotide comprising steps of:
generating a target mRNA vector having an mRNA transcript possessing a first end and a second end and lacking a cap and a poly(A) tail,
wherein a first terminal hairpin structure is located at said first end of said mRNA transcript,
wherein a second terminal hairpin structure is located at said second end of said mRNA transcript,
wherein said first terminal hairpin structure and said second terminal hairpin structure are nucleotide sequences configured to stably fold onto themselves via nucleotide pairing, and
wherein said first terminal hairpin structure and said second terminal hairpin structure are linked to said first end and said second end via no more than two unpaired nucleotides,
wherein said target mRNA vector has one of a mRNA transcript and a chemically synthesized mRNA molecule,
wherein said first terminal hairpin structure has a number of individual hairpins that is at least equal to a number of individual hairpins of said second terminal hairpin structure,
wherein said number of individual hairpins of said first terminal hairpin structure determines an amount of a target mRNA vector product,
inserting said target mRNA vector sequence into a DNA source to create a DNA template,
performing in vitro transcription on said DNA template using DNA-dependent RNA polymerase to obtain a target mRNA vector product, and
purifying said target mRNA vector product to produce therapeutic exogenous mRNA.

77. The method of claim 76, wherein a cap is connected to said mRNA transcript at a 5′ end, wherein said 5′ end is one of said first end or said second end, wherein one of said first terminal hairpin structure or said second terminal hairpin structure is secured downstream to said cap.

78. The method of claim 76, wherein a poly(A) tail is connected to a 3′ end, wherein said 3′ end is one of said first end or said second end, wherein one of said first terminal hairpin structure or said second terminal hairpin structure is secured upstream to said poly(A) tail.

79. The method of claim 76, wherein a cap is connected to said first end and a poly(A) tail is connected to said second end, wherein said first end is a 5′ end and said second end is a 3′ end, wherein said first terminal hairpin structure is secured downstream of said cap and said second terminal hairpin structure is secured upstream of said poly(A) tail.

80. The method of claim 76, wherein said first terminal hairpin structure and said second terminal hairpin structure are one of a triple hairpin structure, double hairpin structure, and single hairpin structure,
wherein said double hairpin structure and said triple hairpin structure comprise no more than two unpaired nucleotides between said individual hairpins.

81. The method of claim 76, further comprising a delivered gene sequence that is incorporated into said mRNA transcript.

82. The method of claim 81, wherein an internal translation initiation site of said target mRNA vector is incorporated into said mRNA transcript.

83. The method of claim 82, wherein said internal translation initiation site is an internal ribosome entry site (IRES).

84. The method of claim 82, wherein said internal translation initiation site is incorporated into said mRNA transcript at a location that is upstream of said delivered gene sequence of said mRNA transcript.

85. The method of claim 84, wherein said internal translation initiation site is incorporated into said mRNA transcript at a 5′ untranslated region (5′ UTR) of said mRNA transcript.

86. The method of claim 76, wherein said DNA source is one of a plasmid DNA, genomic DNA, synthetic DNA, and PCR product.

87. A method for creating a biologically functional polynucleotide comprising steps of:
generating a target mRNA vector having a chemically synthesized mRNA molecule possessing a first end and a second end and lacking a cap and a poly(A) tail,
wherein a first terminal hairpin structure is located at said first end of said chemically synthesized mRNA molecule,
wherein a second terminal hairpin structure is located at said second end of said chemically synthesized mRNA molecule,
wherein said first terminal hairpin structure and said second terminal hairpin structure are nucleotide sequences configured to stably fold onto themselves via nucleotide pairing, and
wherein said first terminal hairpin structure and said second terminal hairpin structure are linked to said first end and said second end via no more than two unpaired nucleotides,
wherein said first terminal hairpin structure has a number of individual hairpins that is at least equal to a number of individual hairpins of said second terminal hairpin structure,
wherein said number of individual hairpins of said first terminal hairpin structure determines an amount of said target mRNA vector,
purifying said target mRNA vector to produce therapeutic exogenous mRNA.

88. The method of claim 87, wherein a cap is connected to said chemically synthesized mRNA molecule at a 5′ end, wherein said 5′ end is one of said first end or said second end, wherein one of said first terminal hairpin structure or said second terminal hairpin structure is secured downstream to said cap.

89. The method of claim 87, wherein said first terminal hairpin structure and said second terminal hairpin structure are one of a triple hairpin structure, double hairpin structure, and single hairpin structure,
wherein said double hairpin structure and said triple hairpin structure comprise no more than two unpaired nucleotides between said individual hairpins.

90. The method of claim 87, further comprising a delivered gene sequence that is incorporated into said chemically synthesized mRNA molecule.

91. The method of claim 90, wherein an internal translation initiation site of said target mRNA vector is incorporated into said chemically synthesized mRNA molecule.

92. The method of claim 91, wherein said internal translation initiation site is an internal ribosome entry site (IRES).

93. The method of claim 91, wherein said internal translation initiation site is incorporated into said chemically synthesized mRNA molecule at a location that is upstream of said delivered gene sequence of said chemically synthesized mRNA molecule.

94. The method of claim 93, wherein said internal translation initiation site is incorporated into said chemically synthesized mRNA molecule at a 5′ untranslated region (5′ UTR) of said chemically synthesized mRNA molecule.

95. A method for creating a biologically functional polynucleotide comprising steps of:
generating a target mRNA vector having an mRNA transcript possessing a 5′ end and a 3′ end and lacks a poly(A) tail,
wherein a first terminal hairpin structure is located at said 3′ end of said mRNA transcript,
wherein a cap is located at said 5′ end of said mRNA transcript,
wherein said first terminal hairpin structure is a nucleotide sequence configured to stably fold onto itself via nucleotide pairing,
wherein a number of individual hairpins of said first terminal hairpin structure determines an amount of a target mRNA vector product,
wherein said first terminal hairpin structure is linked to said 3′ end via no more than two unpaired nucleotides,
inserting said target mRNA vector sequence into a DNA source to create a DNA template,
performing in vitro transcription on said DNA template using DNA-dependent RNA polymerase to obtain a said target mRNA vector product, and
purifying said target mRNA vector product to produce therapeutic exogenous mRNA.

96. The method of claim 95, further comprising a poly(A) tail at said 3′ end, wherein said first terminal hairpin structure is secured upstream to said poly(A) tail.

97. The method of claim 95, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

98. The method of claim 95, further comprising an internal translation initiation site that is incorporated into said mRNA transcript.

99. The method of claim 98, wherein said internal translation initiation site is an internal ribosome entry site (IRES).

100. The method of claim 95, further comprising a delivered gene sequence that is incorporated into said mRNA transcript.

101. The method of claim 100, wherein an internal translation initiation site is incorporated into said mRNA transcript at a location that is upstream of said delivered gene sequence of said mRNA transcript.

102. The method of claim 101, wherein an internal translation initiation site is incorporated into said mRNA transcript at a 5′ untranslated region (5′ UTR) of said mRNA transcript.

103. The method of claim 95, wherein said DNA source is one of a plasmid DNA, genomic DNA, synthetic DNA, and PCR product.

104. A method for creating a biologically functional polynucleotide comprising steps of:
generating a target mRNA vector having an mRNA transcript possessing a 5′ end and a 3′ end and lacking a cap,
wherein a first terminal hairpin structure is located at said 5′ end of said mRNA transcript,
wherein a poly(A) tail is located at said 3′ end of said mRNA transcript,
wherein said first terminal hairpin structure is a nucleotide sequence configured to stably fold onto itself via nucleotide pairing,
wherein a number of individual hairpins of said first terminal hairpin structure determines an amount of a target mRNA vector product,
wherein said first terminal hairpin structure is linked to said 5′ end via no more than two unpaired nucleotides,
inserting said target mRNA vector into a DNA source to create a DNA template,
performing in vitro transcription on said DNA template using DNA-dependent RNA polymerase to obtain a target mRNA vector product, and
purifying said target mRNA vector product to produce therapeutic exogenous mRNA.

105. The method of claim 104, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

106. The method of claim 104, further comprising an internal translation initiation site that is incorporated into said mRNA transcript.

107. The method of claim 106, wherein said internal translation initiation site is an internal ribosome entry site (IRES).

108. The method of claim 107, further comprising a delivered gene sequence that is incorporated into said mRNA transcript.

109. The method of claim 108, wherein said internal translation initiation site is incorporated into said mRNA transcript at a location that is upstream of said delivered gene sequence of said mRNA transcript.

110. The method of claim 109, wherein an internal translation initiation site is incorporated into said mRNA transcript at a 5′ untranslated region (5′ UTR) of said mRNA transcript.

111. The method of claim 104, wherein said DNA source is one of a plasmid DNA, genomic DNA, synthetic DNA, and PCR product.

112. A method for creating a biologically functional polynucleotide comprising steps of:
generating a target mRNA vector having a chemically synthesized mRNA molecule possessing a 5′ end and a 3′ end and lacks a poly(A) tail,
wherein a first terminal hairpin structure is located at said 3′ end of said chemically synthesized mRNA molecule,
wherein a cap is located at said 5′ end of said chemically synthesized mRNA molecule,
wherein said first terminal hairpin structure is a nucleotide sequence configured to stably fold onto itself via nucleotide pairing,
wherein a number of individual hairpins of said first terminal hairpin structure determines an amount of a target mRNA vector product,
wherein said first terminal hairpin structure is linked to said 3′ end via no more than two unpaired nucleotides,
obtaining said target mRNA vector product using said target mRNA vector, and
purifying said target mRNA vector product to produce therapeutic exogenous mRNA.

113. The method of claim 112, further comprising a poly(A) tail at said 3′ end, wherein said first terminal hairpin structure is secured upstream to said poly(A) tail.

114. The method of claim 112, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

115. The method of claim 112, further comprising an internal translation initiation site that is incorporated into said chemically synthesized mRNA molecule.

116. The method of claim 115, wherein said internal translation initiation site is an internal ribosome entry site (IRES).

117. The method of claim 115, further comprising a delivered gene sequence that is incorporated into said chemically synthesized mRNA molecule.

118. The method of claim 117, wherein said internal translation initiation site is incorporated into said chemically synthesized mRNA molecule at a location that is upstream of said delivered gene sequence of said chemically synthesized mRNA molecule.

119. The method of claim 118, wherein said internal translation initiation site is incorporated into said chemically synthesized mRNA molecule at a 5′ untranslated region (5′ UTR) of said chemically synthesized mRNA molecule.

120. A method for creating a biologically functional polynucleotide comprising steps of:
generating a target mRNA vector having a chemically synthesized mRNA molecule possessing a 5′ end and a 3′ end and lacking a cap,
wherein a first terminal hairpin structure is located at said 5′ end of said chemically synthesized mRNA molecule,
wherein a poly(A) tail is located at said 3′ end of said chemically synthesized mRNA molecule,
wherein said first terminal hairpin structure is a nucleotide sequence configured to stably fold onto itself via nucleotide pairing,
wherein a number of individual hairpins of said first terminal hairpin structure determines an amount of target mRNA vector product,
wherein said first terminal hairpin structure is linked to said 5′ end via no more than two unpaired nucleotides,
obtaining a target mRNA vector product using said target mRNA vector, and
purifying said target mRNA vector product to produce therapeutic exogenous mRNA.

121. The method of claim 120, wherein said first terminal hairpin structure is one of a triple hairpin structure, double hairpin structure, and single hairpin structure,
wherein said double hairpin structure and said triple hairpin structure comprise no more than two unpaired nucleotides between said individual hairpins.

122. The method of claim 120, further comprising an internal translation initiation site that is incorporated into said chemically synthesized mRNA molecule.

123. The method of claim 122, wherein said internal translation initiation site is an internal ribosome entry site (IRES).

124. The method of claim 122, further comprising a delivered gene sequence that is incorporated into said chemically synthesized mRNA molecule.

125. The method of claim 124, wherein said internal translation initiation site is incorporated into said chemically synthesized mRNA molecule at a location that is upstream of said delivered gene sequence of said chemically synthesized mRNA molecule.

126. The method of claim 125, wherein said internal translation initiation site is incorporated into said chemically synthesized mRNA molecule at a 5′ untranslated region (5′ UTR) of said chemically synthesized mRNA molecule

127. A product for use as a therapeutic agent, wherein said product is prepared by:
inserting a target mRNA vector sequence into a DNA source to create a DNA template,
wherein said target mRNA vector sequence encodes a first terminal hairpin structure that is located at a first end of a target mRNA vector and a second terminal hairpin structure that is located at a second end of said target mRNA vector,
wherein an internal translation initiation site is incorporated into said target mRNA vector,
wherein said target mRNA vector sequence lacks a cap and a poly(A) tail,
wherein said first terminal hairpin structure has a number of individual hairpins that is at least equal to a number of individual hairpins of said second terminal hairpin structure,
wherein said number of individual hairpins of said first terminal hairpin structure determines an amount of target mRNA vector product,
performing in vitro transcription on said DNA template using DNA-dependent RNA polymerase to obtain a target mRNA vector product, and
purifying said target mRNA vector product to produce a therapeutic agent,
wherein said therapeutic agent is said product.

128. A product for use as a therapeutic agent, wherein said product is prepared by:
inserting a target mRNA vector sequence into a DNA source to create a DNA template,
wherein said target mRNA vector sequence encodes a first terminal hairpin structure onto a 5′ end of a target mRNA vector and a poly(A) tail at a 3′ end of said target mRNA vector,
wherein said target mRNA vector sequence lacks a cap,
wherein a number of individual hairpins of said first terminal hairpin structure determines an amount of a target mRNA vector product,
wherein an internal translation initiation site is incorporated into said target mRNA vector,
performing in vitro transcription on said DNA template using DNA-dependent RNA polymerase to obtain a target mRNA vector product, and
purifying said target mRNA vector product to produce a therapeutic agent,
wherein said therapeutic agent is said product.

129. A product for use as a therapeutic agent, wherein said product is prepared by:
inserting a target mRNA vector sequence into a DNA source to create a DNA template,
wherein said target mRNA vector sequence encodes a first terminal hairpin structure onto a 3′ end of a target mRNA vector and a cap at a 5′ end of said target mRNA vector,
wherein an internal translation initiation site is incorporated into said target mRNA vector,
wherein said target mRNA vector sequence lacks a poly(A) tail,
wherein a number of individual hairpins of said first terminal hairpin structure determines an amount of a target mRNA vector product,
performing in vitro transcription on said DNA template using DNA-dependent RNA polymerase to obtain a target mRNA vector product, and
performing in vitro transcription on said DNA template using DNA-dependent RNA polymerase to obtain a target mRNA vector product, and
purifying said target mRNA vector product to produce a therapeutic agent,
wherein said therapeutic agent is said product.

130. A product for use as a therapeutic agent, wherein said product is prepared by:
performing chemical synthesis on an mRNA vector having a target mRNA vector sequence to create a chemically synthesized mRNA molecule,
wherein said mRNA vector has a first end and a second end,
wherein said target mRNA vector sequence encodes a first terminal hairpin structure onto a first end of a target mRNA vector and a second terminal hairpin structure onto a second end of said target mRNA vector,
wherein an internal translation initiation site is incorporated into said chemically synthesized mRNA molecule,
wherein said target mRNA vector sequence lacks a cap and a poly(A) tail,
wherein said first terminal hairpin structure has a number of individual hairpins that is at least equal to a number of individual hairpins of said second terminal hairpin structure,
wherein said number of individual hairpins of said first terminal hairpin structure determines an amount of a target mRNA vector product,
performing transcription on said chemically synthesized mRNA molecule to obtain a target mRNA vector product,
purifying said target mRNA vector product to produce a therapeutic agent,
wherein said therapeutic agent is said product.

131. A product for use as a therapeutic agent, wherein said product is prepared by:
performing chemical synthesis on an mRNA vector having a target mRNA vector sequence to create a chemically synthesized mRNA molecule,
wherein said target mRNA vector sequence encodes a first terminal hairpin structure at a 5′ end of a target mRNA vector and a poly(A) tail at a 3′ end of said target mRNA vector,
wherein said target mRNA vector sequence lacks a cap,
wherein a number of individual hairpins of said first terminal hairpin structure determines an amount of a target mRNA vector product,
wherein an internal translation initiation site is incorporated into said chemically synthesized mRNA molecule,
performing transcription on said chemically synthesized mRNA molecule to obtain a target mRNA vector product,
purifying said target mRNA vector product to produce a therapeutic agent,
wherein said therapeutic agent is said product

132. A product for use as a therapeutic agent, wherein said product is prepared by:
performing chemical synthesis on an mRNA vector having a target mRNA vector sequence to create a chemically synthesized mRNA molecule,
wherein said target mRNA vector sequence encodes a first terminal hairpin structure at a 3′ end of a target mRNA vector and a cap at a 5′ end of said target mRNA vector,
wherein an internal translation initiation site is incorporated into said chemically synthesized mRNA molecule,
wherein said target mRNA vector sequence lacks a poly(A) tail,
wherein a number of individual hairpins of said first terminal hairpin structure determines an amount of a target mRNA vector product,
performing transcription on said chemically synthesized mRNA molecule to obtain a target mRNA vector product,
purifying said target mRNA vector product to produce a therapeutic agent,
wherein said therapeutic agent is said product.
